(19) United States
(12) Patent Application Publication
(10) Pub. No.: US 2013/0204155 A1
(43) Pub. Date: Aug. 8, 2013

(54) Anesthesia Monitoring Systems and Methods of Monitoring Anesthesia

(71) Applicants: T4 Analytics LLC, Dover, DE (US); Mayo Foundation For Medical Education And Research, Rochester, MN (US)

(72) Inventors: Sorin Joseph Brull, Ponte Vedra Beach, FL (US); David Robert Hampton, Woodinville, WA (US)

(73) Assignees: MAYO FOUNDATION FOR MEDICAL EDUCATION AND RESEARCH, Rochester, MN (US); T4 ANALYTICS LLC, Dover, DE (US)

(21) Appl. No.: 13/750,463

(22) Filed: Jan. 25, 2013

Related U.S. Application Data
(60) Provisional application No. 61/591,547, filed on Jan. 27, 2012.

(51) Int. Cl. A61B 5/00; A61B 5/0488

(52) U.S. Cl. CPC A61B 5/4821; A61B 5/0488
USPC 600/546

(57) ABSTRACT

Provided are systems, devices and methods for monitoring anesthesia. For example, the methods, devices and systems are optionally used to assess neuromuscular blockade in a subject who has received a muscle relaxant (NMBA) agent.

### CROSS-REFERENCE TO RELATED APPLICATIONS

This application claims the benefit of U.S. Provisional Patent Application No. 61/591,547, filed on Jan. 27, 2012, entitled “Anesthesia Monitoring Systems and Methods of Monitoring Anesthesia,” the disclosure of which is expressly incorporated herein by reference in its entirety.

### TECHNICAL FIELD

This document relates to monitoring and assessment of anesthesia levels in subjects.

### BACKGROUND

About 230 million surgeries take place annually world-wide; 40 million US patients undergo in-hospital general anesthesia, which induces loss of consciousness, each year, and 25 million of those also receive muscle relaxants (also called neuromuscular blocking agents, NMBAs), which inhibit neuromuscular transmission. These relaxant agents decrease muscle tension and suppress reflex contractions, and may be administered for several reasons including the following.

General anesthesia requires that patients' lungs be mechanically (artificially) ventilated via an endotracheal tube (breathing tube, or ETT) that is placed into the trachea (wind-pipe). This tube must pass down the throat and between the vocal cords, and muscle relaxants make this procedure possible and safer for patients.

Surgeries involving the abdomen, the lungs and the brain require muscle relaxants (neuromuscular blocking drugs, NMBAs) to allow the surgeon to work and to minimize injury to the patient and to the organs.

Secondary applications include gynecologic, orthopedic, plastic surgery and laparoscopic procedures, and various procedures performed in intensive care unit (ICU), emergency department (ED) and Ambulatory Care Center (ACC); these procedures require neuromuscular blockade and mechanical ventilation.

Muscle relaxants (NMBAs) have two forms: depolarizing agents, which are short-acting (5-10 min duration) and are sometimes used at the start of anesthesia to facilitate tracheal intubation, and non-depolarizing agents that have a longer duration of action (20-60 min), and that are used to maintain muscle relaxation during surgery. The effects of non-depolarizing agents start within minutes and continue for up to 20-60 minutes after withdrawal (depending on the type of relaxant used), so they must be administered repeatedly throughout the surgical procedure.

Drug effects must completely dissipate once the surgical procedure is complete, however, so that patients can start breathing on their own (spontaneously). Reversal drugs (anticholinesterases) can be administered to speed-up recovery from muscle relaxants, but reversal drugs can slow the heart to dangerous levels (bradycardia), and can have a host of other unpleasant side effects, such that atropine (or glycopyrrolate) is commonly administered as an adjunct to reversal agents. Unfortunately, atropine and atropine-like agents also have their own additional side-effects, such as nausea, vomiting and tachycardia.

Overdosing of relaxants to assure complete muscle paralysis during surgery can lead to delayed recovery of muscle function, prolonging recovery room stays, hospital stays and increasing healthcare costs. 30-60% of patients admitted to the postoperative care unit (Recovery Room, or PACU) have significant residual muscle weakness (i.e., incomplete reversal of paralysis). In extreme cases, patients can experience a Critical Respiratory Event (CRE) in which they are unable to breathe independently. CRE affects 0.8% of patients who have residual weakness, and may require emergency placement of another breathing tube; approximately 10,000 patients are estimated to require emergent re-insertion of the breathing tube each year from complications of post-surgical CRE. The need for emergent reintubation leads to morbidity and mortality, and markedly increases the cost of healthcare.

An optimal dose of paralytic (muscle relaxant) medications should be based on the effect that they have on muscles, rather than dosing based on physical characteristics of the patient (age, sex, weight) or drug concentration (blood or tissue). Unfortunately, simple subjective assessment of muscle tone, spontaneous breathing, and reflex responses are not accurate or consistent indicators of relaxant effect.

Neuromuscular Monitors have been proposed to give more precise indication of degree of neuromuscular block, but these are hard to use and expensive—in fact, less than 25% of American anesthesiologists use nerve stimulators to test muscle function, and no more than 5% of American anesthesiologists objectively measure the state of muscle reversal.

Accelerographs and mechanomyographs are neuromuscular monitors that are sometimes used to measure the twitch of muscle as it contracts in response to an electrical stimulus applied to a nerve. However, accurate measurement of the strength of amplitude of twitch, corresponding to the degree of neuromuscular block, is difficult: piezoelectric sensors (that accelerographs are based on) give variable results and can be masked by artifact from other movements. The visible response may disappear before full relaxation is achieved and the technique cannot be applied to smaller muscles on the face. The monitors demonstrate hysteresis, in that the measured values do not fully return to baseline values once blockade is reversed. Accelerometers also require full access to the muscle being monitored (usually the thumb), but the patients' arms may be unavailable to the anesthesiologist in a significant (40-70%) number of cases, as the patients' arms are often tucked under the surgical drapes. Mechanomyographs have fewer problems with hysteresis, but are difficult to place on the patient, are relatively bulky, they require access to the thumb for monitoring, and therefore cannot be used in surgical procedures that require a patient's arms to be tucked under the surgical drapes. Currently, there are no mechanomyographs available for purchase or for clinical use in the United States. The few remaining mechanomyographs were built in the 1990s and are used in a handful of research centers.

### SUMMARY

Provided are systems, devices and methods for monitoring neuromuscular blockade of muscles in patients being administered muscle relaxants. The muscle relaxant agent is optionally a neuromuscular blocking agent. Optionally, the muscle relaxant agent is a depolarizing agent. Optionally, the muscle relaxant agent is a non-depolarizing agent.

An example method for assessing neuromuscular blockade in a subject having been administered a muscle relaxant agent includes stimulating a motor nerve with a plurality of temporally distinct stimuli. After each stimulus of the motor nerve, the muscle response in the muscle(s) innervated by the stimulated motor nerve is recorded. The recorded muscle responses following the application of the plurality of stimuli are evaluated to provide an assessment of neuromuscular blockade in the subject. Each stimulus of the plurality is sufficient to cause an evoked muscle response under normal physiological conditions. As muscle relaxants are administered to patients, the evoked muscle response decreases. Evaluation of the muscle responses optionally includes determining a ratio of an amplitude of a particular recorded muscle response to the amplitude of a muscle response resulting from any subsequent or previous stimulus to characterize the degree of decrement, which is related to the degree of blockade. In some implementations, evaluation of the muscle responses may include determining a ratio of the amplitude of a muscle response from a subsequent stimulus to the amplitude of the muscle response from a previous stimulus. A determined ratio less than 1.0 indicates the presence of neuromuscular blockade in the subject, for example.

Optionally, the first stimulus produces an evoked muscle response. Optionally, one or more of the subsequent stimuli do not produce an evoked muscle response. When the subsequent and/or first stimulus does not produce an evoked muscle response, the determined ratio is zero indicating presence of neuromuscular blockade in the subject.

Optionally, as a supplemental measure, the number of subsequent stimuli evoking a muscle response may be counted, allowing assessment of degree of blockade when the determined ratio is zero.

Optionally, the ratio is determined as a ratio of the amplitude of the muscle response related to the fourth stimulus to the amplitude of the muscle response related to the first stimulus of a plurality of stimuli that includes at least four stimuli. Although it is not required, in some implementations, the fourth stimulus may be the fourth sequential stimulus and the first stimulus may be the first stimulus in the plurality of sequential stimuli. Optionally, the ratio is determined as a ratio of the amplitude of the muscle response related to the fifth or greater stimulus to the amplitude of the muscle response related to the first stimulus. For example, the ratio is optionally determined from the amplitude of the muscle response related to the sixth, seventh, eighth, ninth, or tenth stimulus to the amplitude of the muscle response related to the first stimulus. Regardless of which number subsequent stimulus is used, the ratio is zero if there is no muscle response related to the first and/or the subsequent stimulus of the plurality.

The method optionally further includes identifying one or more stimuli of the plurality of temporally distinct stimuli that caused an evoked muscle response and enumerating them to determine a count. The count can be determined subsequent to determining a zero value ratio. Optionally, the count is zero. A count of zero indicates that none of the one or more of the plurality of stimuli used to determine the count caused an evoked muscle response.

If the ratio or the count is zero, the method optionally comprises stimulating the motor nerve in a tetanic protocol. A tetanic protocol may optionally comprise delivering a plurality of stimuli at a rate that is high enough to cause fusion of the individual evoked muscle responses into a single sustained muscle contraction. Optionally, this may be a rate greater than 30 stimuli per second. A ratio of the amplitude of the last evoked response to the amplitude of the first evoked response may be calculated, and a value less than 1.0 demonstrates the presence of neuromuscular block. Alternatively, because there may be some amplitude variation in the evoked muscle responses at the beginning of the tetanic stimulation, a ratio of the amplitude of any response toward the end of the stimulation to the amplitude of any response toward the beginning of the stimulation may be calculated, and a value less than 1.0 demonstrates the presence of neuromuscular block. For example, there may be some amplitude variation in the evoked responses during the first 1-3 seconds of the stimulation. In some implementations, the response towards the beginning of the stimulation with the largest amplitude may be used in the ratio.

Subsequent to administering the tetanic protocol, the motor nerve is optionally stimulated with a plurality of temporally spaced supplemental stimuli. After each stimulation of the motor nerve, the muscle responses of the muscle innervated by the stimulated motor nerve are recorded. The number of evoked muscle responses produced by the temporally spaced subsequent stimuli is used to determine a post-tetanic count and indicates a degree of neuromuscular block.

Another example method for assessing neuromuscular blockade in a subject having been administered a muscle relaxant agent includes stimulating a motor nerve to cause an evoked muscle response. The evoked muscle response is recorded. A peak of the recorded evoked muscle response is identified. The amplitude of the peak from baseline is determined. The measured amplitude from baseline is compared to a control amplitude, determined from prior stimuli, to indicate a change in the level of neuromuscular blockade in the subject or to demonstrate that the desired level of blockade has been maintained.

For purposes of determining the amplitude of the evoked muscle response, the start of the evoked muscle response is optionally identified by locating the time at which the level of muscle electrical activity exceeds the baseline level of electrical noise in the subject prior to stimulation of the motor nerve or immediately following completion of the evoked response. The baseline level of electrical noise is the background noise and may include all ambient, intrinsic and extrinsic noise. The amplitude is determined as the difference in measured voltage from the baseline level to the positive or negative peak of the evoked muscle response caused by stimulating the motor nerve.

Optionally, the control amplitude is the amplitude determined from the baseline level to the positive or negative peak of a prior or subsequent evoked muscle response caused by stimulating the same motor nerve. Optionally, the control amplitude is determined as the amplitude of a prior or subsequent evoked muscle response produced during a repeated train of stimuli to the motor nerve. The repeated train of stimuli optionally includes four stimuli, each stimulus capable to cause an evoked muscle response. For example, the repeated train of stimuli may be a train-of-four stimulation protocol. In another example, the repeated train of stimuli is produced during a tetanic stimulation protocol of the motor nerve.

If a train-of-four protocol is used, optionally, one or more of the four stimuli do not produce a corresponding evoked muscle response. Optionally, the train of stimuli produces a plurality of evoked muscle responses. A peak of one or more of the plurality of evoked muscle responses is optionally identified and its amplitude is determined. An identified peak optionally has the largest negative value in its corresponding evoked muscle response. An identified peak optionally has the largest positive value in its corresponding evoked muscle response.

Optionally, the control amplitude is the amplitude determined from the baseline level to peak amplitude of the evoked muscle response caused by the first stimulation. In other implementations, the control amplitude may be the amplitude determined from the baseline level to peak amplitude of any prior or subsequent muscle response. The control amplitude may be the denominator in the ratio of amplitudes and the determined amplitude may be the numerator in the ratio of amplitudes. For example, when the evoked muscle response caused by the first stimulation is used as the control, the comparison of the determined amplitude corresponding to the first stimulus and the amplitude corresponding to the fourth stimulus includes determining a ratio of the amplitude corresponding to the fourth stimulus and the amplitude corresponding to the first stimulus. Although not required, in some implementations, the fourth stimulus may be the fourth sequential stimulus and the first stimulus may be the first stimulus in the plurality of sequential stimuli.

Optionally, the control amplitude is the amplitude determined from the baseline to peak of the evoked muscle response caused by the first, second, third, fourth or a subsequent stimulation. The control amplitude optionally has a value of zero. When the control amplitude, whether the first, second, third, fourth, or a subsequent evoked muscle, is zero, the method optionally further comprises determining if there was an evoked muscle response corresponding to the first, second, third stimuli, or if there was an evoked muscle response corresponding to any predetermined stimulus preceding the final stimulus of a given stimulus protocol.

A count number representing the count of the number of evoked muscle responses corresponding to the number of first, second and third stimuli that elicit non-zero amplitude responses is optionally displayed. If there is no evoked muscle response corresponding to the first, second and third stimuli the count is zero and the motor nerve is optionally stimulated using a tetanic protocol.

Another example method for assessing neuromuscular blockade in a subject having been administered a muscle relaxant agent includes stimulating a motor nerve to cause an evoked muscle response and recording the evoked muscle response. A first peak of the recorded evoked muscle response and a subsequent peak of opposite polarity in the recorded evoked muscle response are identified. The peak-to-peak amplitude is calculated by identifying the difference between the first and subsequent peak. The determined peak-to-peak amplitude compared to a control peak-to-peak amplitude indicates a level of neuromuscular blockade in the subject.

The control amplitude is optionally the amplitude determined from the peak-to-peak amplitude between the first and subsequent peak of opposite polarity in a prior or subsequent evoked muscle response caused by stimulating the same motor nerve. The prior or subsequent evoked muscle response is optionally produced during a repeated train of stimuli to the motor nerve such as a train-of-four stimulus protocol or a tetanic stimulus protocol.

When a train-of-four protocol is used, one or more of the four stimuli optionally do not produce a corresponding evoked muscle response. Optionally, the train of stimuli produces a plurality of evoked muscle responses. A peak of one or more of the plurality of evoked muscle responses is optionally identified.

Optionally, the control amplitude has a value of zero. If a train-of-four protocol is used, the determined amplitude corresponding to the first stimulus and the amplitude corresponding to the fourth stimulus can be compared by determining a ratio of the amplitude corresponding to the fourth stimulus to the amplitude corresponding to the first stimulus. The amplitude of the fourth stimulus optionally has a value of zero, which results in a ratio of zero.

If the determined ratio for the train-of-four protocol is zero, optionally it is determined if there was an evoked muscle response corresponding to the first, second and third stimuli. If evoked muscle response is detected related to the first, second, third and fourth stimuli, the motor nerve is optionally stimulated using a tetanic protocol. Subsequent to stimulating the motor nerve using the tetanic protocol, the motor nerve is optionally stimulated with a plurality of temporally spaced stimuli. After stimulating the motor nerve, the muscle responses in the muscle innervated by the stimulated motor nerve are recorded and the number of evoked muscle responses produced by temporally spaced subsequent stimuli is identified.

Another example method for assessing neuromuscular blockade in a subject having been administered a muscle relaxant agent includes stimulating a motor nerve with a plurality of temporally spaced stimuli and recording for muscle electrical activity in the muscle innervated by the stimulated motor nerve after at least two stimulations of the motor nerve.

Peaks of one or more recorded evoked muscle responses are identified. The amplitude of each identified peak from baseline is determined and the amplitude of a peak associated with an evoked muscle response caused by one of the stimuli of the motor nerve is compared to the electrical activity of the muscle after stimulation of the motor nerve with a subsequently applied stimulus. The comparison is used to indicate a level of neuromuscular blockade in the subject.

Optionally, the subsequently applied stimulus causes an evoked muscle response. Optionally, a peak of the evoked muscle response caused by the subsequently applied stimulus is identified and its amplitude determined. Optionally, the amplitude of the peak of the first and subsequent recorded evoked muscle response are compared, wherein a smaller amplitude of the subsequent recorded response as compared to the first evoked muscle response indicates a level of neuromuscular blockade in the subject.

Another example method for assessing neuromuscular blockade in a subject having been administered a muscle relaxant agent includes stimulating a motor nerve to cause a first and a subsequent evoked muscle responses and recording the first and subsequent evoked muscle responses. A peak of the first and subsequent recorded evoked muscle responses are identified and a subsequent peak of the first and subsequent recorded evoked muscle responses are identified. The peak-to-peak amplitude of the first recorded evoked muscle response and the peak-to-peak amplitude of the subsequent recorded evoked muscle response are determined. The peak-to-peak amplitudes are compared, wherein a smaller peak-to-peak amplitude of the subsequent recorded response as compared to the first evoked muscle response indicates a level of neuromuscular blockade in the subject.

Another example method for assessing neuromuscular blockade in a subject having been administered a muscle relaxant agent includes stimulating a motor nerve a first, second, third and fourth time and recording for muscle electrical activity in a muscle innervated by the stimulated motor nerve subsequent to each stimulation of the motor nerve. The muscle electrical response caused by the first and fourth stimuli is quantified and a ratio is determined of the quantified muscle electrical responses caused by the fourth and first stimuli. The determined ratio indicates a level of neuromuscular blockade in the subject. Optionally, each stimulus is sufficient to cause an evoked muscle response. Optionally, each stimulus is temporally distinct. Optionally, the first stimulus causes an evoked muscle response. Optionally, the second stimulus causes an evoked muscle response. Optionally, the third stimulus causes an evoked muscle response. Optionally, the fourth stimulus causes an evoked muscle response. Optionally, the first stimulus does not cause an evoked muscle response. Optionally, the second stimulus does not cause an evoked muscle response. Optionally, the third stimulus does not cause an evoked muscle response. Optionally, the fourth stimulus does not cause an evoked muscle response. Optionally, the determined ratio is zero.

For example, the ratio optionally includes the response to the fourth stimulus, which does not cause an evoked muscle response and the determined ratio is zero. Optionally, the ratio is determined from the muscle response of the fourth and first stimuli in a train-of-four sequence.

If the ratio is zero, it is optionally determined if an evoked muscle response was caused by one or more of the third, second or first stimuli. If none of the third, second or first stimuli produce an evoked muscle response, a series of stimuli is optionally applied to the motor nerve using a tetanic stimulation protocol. Optionally, the tetanic stimulation protocol does not cause an evoked muscle response for the full duration of the plurality of stimuli (or for all stimuli in the tetanic series) and a ratio for the tetanic stimulation protocol is zero.

Another method for determining a train of four ratio in a subject having been administered a neuromuscular blocking agent includes stimulating a motor nerve a first, second, third and fourth time and recording electrical activity in a muscle innervated by the stimulated motor nerve subsequent to each stimulation of the motor nerve. The muscle electrical response caused by the first and fourth stimuli is quantified and the train of four ratio of the quantified muscle electrical responses caused by the fourth and first stimuli is determined. Optionally, each stimulus is sufficient to cause an evoked muscle response. Optionally, each stimulus is temporally distinct.

Optionally, quantification of the muscle electrical response caused by the stimuli comprises identifying a peak of a recorded evoked muscle response and determining the amplitude of the peak from baseline.

Optionally, quantification of the muscle electrical response caused by the stimuli comprises identifying a peak of a recorded evoked muscle response, identifying a subsequent peak of opposite polarity of a recorded evoked muscle response and determining the peak-to-peak amplitude by determining the difference between the first and subsequent peak. Optionally, the identified peak is subsequent to the first peak. Optionally, the peak identified is the peak having the largest negative value in its corresponding evoked muscle response. Optionally, the peak identified is the peak having the largest positive value in its corresponding evoked muscle response. Optionally, the subsequent peak is the next peak of opposite polarity following the first peak in its corresponding evoked muscle response. Optionally, the subsequent peak is the peak with the largest value of opposite polarity following the first peak in its corresponding evoked muscle response.

An example system for assessing neuromuscular blockade in a subject having been administered a muscle relaxant agent includes a stimulator configured for stimulating a motor nerve with a plurality of temporally distinct stimuli and a recorder for recording of a muscle response in the muscle innervated by the stimulated motor nerve. The system further includes at least one processor for evaluating the muscle response following the application of a plurality of the stimuli to provide an assessment of neuromuscular blockade in the subject. Optionally, each stimulus is sufficient to cause an evoked muscle response under normal conditions.

The at least one processor is optionally configured to evaluate the muscle response by determining a ratio of the muscle response resulting from a subsequent and first stimulus. Optionally, the first stimulus produces an evoked muscle response. Optionally, the subsequent stimulus does not produce an evoked muscle response. Optionally the determined ratio is zero (or less than 1.0) indicating a level of neuromuscular blockade in the subject.

The at least one processor is optionally configured to determine the ratio from the muscle response related to the first stimulus and the muscle response related to the fourth stimulus. Optionally, the at least one processor is configured to determine the ratio from the muscle response related to the first stimulus and the muscle response related to fifth or greater stimulus. For example, the at least one processor is optionally configured to determine the ratio from the muscle response related to the first and the muscle response related to the sixth, seventh, eighth, ninth, or tenth stimulus. The determined ratio is optionally zero (or less than 1.0).

The at least one processor is optionally further configured to identify one or more stimuli of the plurality of temporally distinct stimuli that caused an evoked muscle response to determine a count. Optionally, the count is zero indicating that none of the plurality of temporally distinct stimuli caused an evoked muscle response.

The stimulator is optionally further configured to stimulate the motor nerve in a tetanic protocol and to stimulate the motor nerve with a plurality of temporally spaced stimuli subsequent to stimulating the motor nerve with the tetanic protocol. Optionally, the recorder is further configured to record for an evoked muscle response related to each temporally spaced subsequent stimulation and the at least one processor is further configured to identify the number of evoked muscle responses produced by temporally spaced subsequent stimuli to determine a second count.

Another example system for assessing neuromuscular blockade in a patient having been administered a muscle relaxant agent includes a stimulator configured to generate a stimulus to a motor nerve a first, second, third and fourth time. The system further includes a patient-stimulus interface configured to supply each stimulus generated by the stimulator to the patient and a patient-recording interface configured to record muscle electrical activity in a muscle innervated by the stimulated motor nerve subsequent to each stimulation of the motor nerve. The system further includes at least one processing device configured to quantify the muscle electrical response caused by the first and fourth stimuli and to determine a ratio of the quantified muscle electrical responses caused by the fourth and first stimuli. The ratio indicates a level of neuromuscular blockade in the subject.

Optionally, the processing device is further configured to determine if the first, second or third stimuli caused evoked muscle responses. Optionally, the processing device is further configured to cause the stimulator to generate a plurality of stimuli for tetanic stimulation if the first, second, third and fourth stimuli do not cause evoked muscle responses. Optionally, each stimulus is sufficient to cause an evoked muscle response under normal conditions. Optionally, each stimulus is temporally distinct. Each stimulus may be temporally distinct when the stimulus ends (i.e., no more current is delivered), and not necessarily when the muscle response to the stimulus is complete.

An example system for determining a train-of-four ratio includes a stimulator configured to produce stimuli for provision to a motor nerve a first, second, third and fourth time and a recording apparatus configured to record muscle electrical activity in a muscle innervated by the stimulated motor nerve subsequent to each stimulation of the motor nerve. The system further includes at least one processor configured to quantify the muscle electrical response caused by the first and fourth stimuli and for determining the train-of-four ratio of the quantified muscle electrical responses caused by the first and fourth stimuli. Optionally, the at least one processor is configured to quantify the muscle electrical response caused by the stimuli by identifying a peak of a recorded evoked muscle response and determining the amplitude of the peak from baseline. Optionally, the at least one processor is configured to quantify the muscle electrical response caused by the stimuli by identifying a peak of the recorded evoked muscle response, identifying a subsequent peak of the recorded evoked muscle response and determining the peak-to-peak amplitude by determining the difference between the first and subsequent peak. Optionally, each stimulus is sufficient to cause an evoked muscle response. Optionally, each stimulus is temporally distinct.

Also provided is a system for assessing muscle electrical activity in a subject including a motor nerve stimulator configured to stimulate a motor nerve and a recording apparatus for recording electrical activity of a muscle innervated by the motor nerve. The system further includes a recording apparatus for recording electrical activity of the stimulated motor nerve.

The details of one or more embodiments of the invention are set forth in the accompanying drawings and the description below. Other features, objects, and advantages of the invention will be apparent from the description and drawings, and from the claims.

### DESCRIPTION OF DRAWINGS

The components in the drawings are not necessarily to scale relative to each other. Like reference numerals designate corresponding parts throughout the several views.

FIG. 1 is a simplified block diagram illustrating a system for monitoring neuromuscular function during anesthesia;

FIG. 2 illustrates a flow diagram of example operations performed within the system of FIG. 1;

FIG. 3 illustrates a flow diagram of example operations when placing electrodes;

FIG. 4 illustrates a flow diagram of example operations when determining a stimulation current and choosing a stimulation protocol;

FIG. 5 illustrates a flow diagram of example operations when monitoring neuromuscular block;

FIGS. 6A-6F illustrate data collected in response to a nerve stimulus;

FIG. 7 illustrates a flow diagram of example operations when determining the validity of collected data;

FIG. 8 illustrates a flow diagram of example operations when applying the train-of-four (TOF) test protocol;

FIG. 9 illustrates a flow diagram of example operations when applying the tetanic test protocol;

FIG. 10 illustrates a flow diagram of example operations when applying the post-tetanic count (PTC) test protocol;

FIG. 11 illustrates a flow diagram of example operations when monitoring neuromuscular block during an example surgery;

FIG. 12 illustrates a flow diagram of example operations when performing the TOF test protocol prior to intubation;

FIG. 13 illustrates a flow diagram of example operations when performing the tetanic test protocol during surgery and the TOF test protocol after administering reversal drugs;

FIG. 14 illustrates a flow diagram of example operations when turning off the monitoring device;

FIG. 15 illustrates a flow diagram of example operations when removing electrodes; and

FIG. 16 illustrates an example processing device.

### DETAILED DESCRIPTION

Provided are systems, devices and methods for monitoring anesthesia. For example, the methods, devices and systems are optionally used to assess neuromuscular blockade in a subject who has received a muscle relaxant agent. The muscle relaxant agent is optionally a neuromuscular blocking agent. Optionally, the muscle relaxant agent is a depolarizing agent. Optionally, the muscle relaxant agent is a non-depolarizing agent.

The disclosed systems, devices and methods provide an objective measure of nerve and muscle function that corresponds directly to effects that the muscle relaxant agent has on the body. Relaxants can thus be more effectively administered and reversed, providing more precise control over induction of anesthesia and relaxation, and identifying when surgical procedures can be started safely. Periodic muscle function monitoring can also guide the titration of muscle relaxants during the surgery to avoid over- and under-dosing, and can signal when a patient has adequately responded so that the endotracheal (breathing) tube can be introduced (at the beginning of the surgical procedure) or withdrawn (at the end of surgical procedure).

The systems, devices and methods are optionally used to objectively measure the depth of neuromuscular blockade accurately and continuously throughout surgical procedures. The neuromuscular function is directly assessed by comparing the evoked muscle response (the evoked electrical activity behind the muscle “twitch”) in response to electrical stimulation of the corresponding motor nerve. Adequate muscle relaxation has been achieved when the muscle response to repetitive stimulation is extinguished while nerve conduction remains intact. The device repeats the assessment when manually or automatically triggered (at user-selected intervals), providing ongoing monitoring of neuromuscular function status throughout any procedure, using any peripheral motor nerve. Battery-powered, easily applied, clearly visible and shaped to integrate comfortably into the operative setting, the device is the reliable objective monitor that assures controlled drug delivery and appropriate return of neuromuscular function to ensure appropriate surgical conditions thus improving patient safety.

As discussed above, muscle relaxants are administered during some types of surgeries. Muscle relaxants interrupt the chemical conduction across the neuromuscular junction, but do not affect the electrical conduction in either the nerve or the muscle fibers. In particular, the muscle relaxants block receptor sites, which prevent chemical messengers from initiating an electrical response in the muscle fiber. As more receptor sites are blocked, fewer muscle fibers receive stimulation, and both the visible mechanical twitch and the underlying electrical response in the muscle decrease. A single administration of muscle relaxants causes a rapid decrease in the response of the muscle, which then restores to normal over time as the drug is metabolized and then excreted by the body (spontaneous recovery). For a particular dose of muscle relaxant, the magnitude of decrease of the muscle response depends on both the time since drug administration and the muscle that is being monitored. For example, the thumb muscle is affected to a greater degree for the same dose of muscle relaxants than the diaphragm. Successful monitoring, therefore, depends both on identifying the correct muscle, and on continuous monitoring of the evolving effect of muscle relaxant administration and withdrawal (reversal).

Prior to administering the muscle relaxants to the patient, a nerve impulse evoked by the stimulation travels to the muscle and elicits an electrical response that results in a muscle twitch. As the muscle relaxants are applied, the receptor sites are blocked and only some muscle fibers respond. Thus, although the nerve response remains unchanged in strength, the amplitude of the muscle response diminishes, an effect more pronounced in the muscle twitch than in the electrical recording. At full block, all muscle responses are abolished, but the nerve response is preserved. Thus, it is possible to detect a procedural error in the case where the stimulus is moved distant to the nerve, because in such a case, there will be neither a detected nerve response nor a muscle response (twitch).

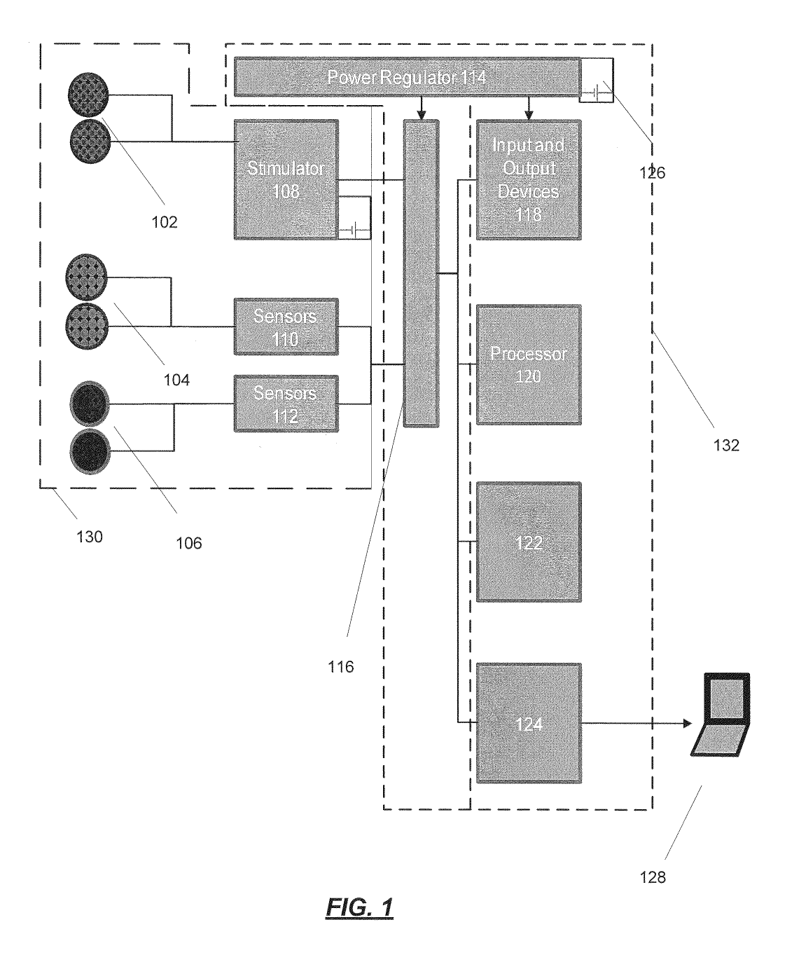

Referring to FIG. 1, a system used to assess the depth of muscle paralysis and degree of muscle recovery in patients receiving muscle relaxants during surgical procedures is described. The system may consist of a stimulating/recording unit 130 and a control/visualization unit 132. The stimulating/recording unit 130 and the control/visualization unit 132 may be connected by a cable or a wireless link. Additionally or alternatively, the stimulating/recording unit 130 and the control/visualization unit 132 may be combined into a single package. However, the effect of electrical noise (i.e., electrocautery) and the physical inconvenience of having additional wires alongside the patient can be minimized if the stimulating/recording unit 130 and the control/visualization unit 132 are physically separated. When physically separated, the stimulating/recording unit 130 and the control/visualization unit 132 may be separate, single, hand-held packages. In addition, the control/visualization unit 132 may be lightweight, textured along the edges but without sharp corners or projecting surfaces. The control/visualization unit 132 may be capable of sitting on a flat surface or being attached to an IV pole and may be constructed of materials with colors that fit operating room standards (i.e., blue and silver). In addition, the control/visualization unit 132 may be amenable to cleaning and sterilization with a damp cloth or alcohol wipe. The control/visualization unit 132 may also be capable of surviving repeated drops from approximately four feet onto a hard floor.

The stimulating/recording unit 130 may include a nerve stimulator 108 and sensors 110 and 112, which may optionally be integrated into a single, hand-held package. The nerve stimulator 108 is capable of delivering electrical pulses to a motor nerve such as the median or ulnar nerve at the wrist, the tibial nerve at the ankle or the facial nerve beneath the ear, for example. In one implementation, the nerve stimulator 108 may deliver a 200 μs or 300 μs square-wave, monophasic, constant electrical pulse. The electrical pulse delivered by the nerve stimulator 108 should be sufficient in strength to elicit nerve responses when the patient is in an unblocked state. In addition, the nerve stimulator 108 may be capable of delivering sequences of pulses, for example train-of-four (TOF) and tetanic bursts.

The sensors 110 and 112 are capable of sensing the intrinsic electrical activity of the nerve and muscle, which are induced by the nerve stimulation. By sensing the electrical activity of the muscle, for example, it is possible to measure the amplitude of the electrical activity, which directly corresponds to the strength of the muscle response. Accordingly, it is possible to determine the impact that the muscle relaxants have on the patient at any point in time during the surgery because changes in the amplitude of the electrical activity of the muscle can be correlated directly to changes caused by addition and reversal of the muscle relaxants.

Stimulating electrodes 102 and sensing electrodes 104 and 106 may be attached to the stimulating/recording unit 130 using a custom connector, for example.

The system shown in FIG. 1 may also include a power regulator 114 that supplies power to both the stimulating/recording unit 130 and the control/visualization unit 132. In some implementations, however, the stimulating/recording unit 130 and the control/visualization unit 132 may be separately powered (i.e., by two separate battery packs). The stimulating/recording unit 130 and the control/visualization unit 132 may be isolated from each other using a galvanic separator 116, for example, to prevent direct current from flowing between the stimulating/recording unit 130 and the control/visualization unit 132.

The control/visualization unit 132 may contain user-input controls and a visual display, store operating protocols, collect patient data and generate a system clock. For example, the control/visualization unit 132 may include input and output devices 118, a processing device 120, an IV-pole holder 122 and an external communication link 124. The input and output devices 118 may include user-input controls such as, for example, a power on/off control, a test protocol selection control (single twitch, Train of Four (TOF), tetanic, Post-tetanic count (PTC)), a stimulus intensity control (0-100 mA constant current), a stimulus mode control (manual or continuous), a stimulus trigger control, etc. The user-input controls may consist of backlit buttons for indicating active modes and successful selections, and audible tones may optionally be used for alarms. In addition, the user-input controls may be designed such that the user can operate the controls while wearing surgical gloves.

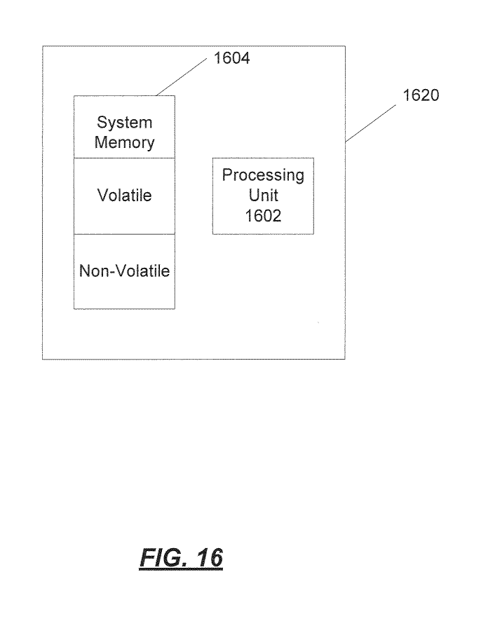

The input and output devices 118 may include a display. For example, the display may be capable of displaying a visual indicator that the control/visualization unit 132 is on, fault indicators (i.e., low battery, loss of electric continuity, failure to deliver stimulus, loss of communication connection), stimulus intensity, bar graphs representing responses to the stimuli, etc. The display is not limited to the visual indicators listed above, and instead may consist of a number of combinations of visual indicators that allow the user to more easily operate the system. The system shown in FIG. 1 may also include a processing device 120 for implementing aspects described herein. An example processing device is discussed in detail below with regard to FIG. 16.

In addition, the system shown in FIG. 1 may include an IV-pole holder 122 and an external communication link 124. The IV-pole holder 122 may be used for securing the control/visualization unit 132 to the IV pole during the surgery. The external communication link 124 allows the system to communicate with other devices. At the conclusion of the surgery, it may be possible to download the collected data from the control/visualization unit 132 using the external communication link 124.

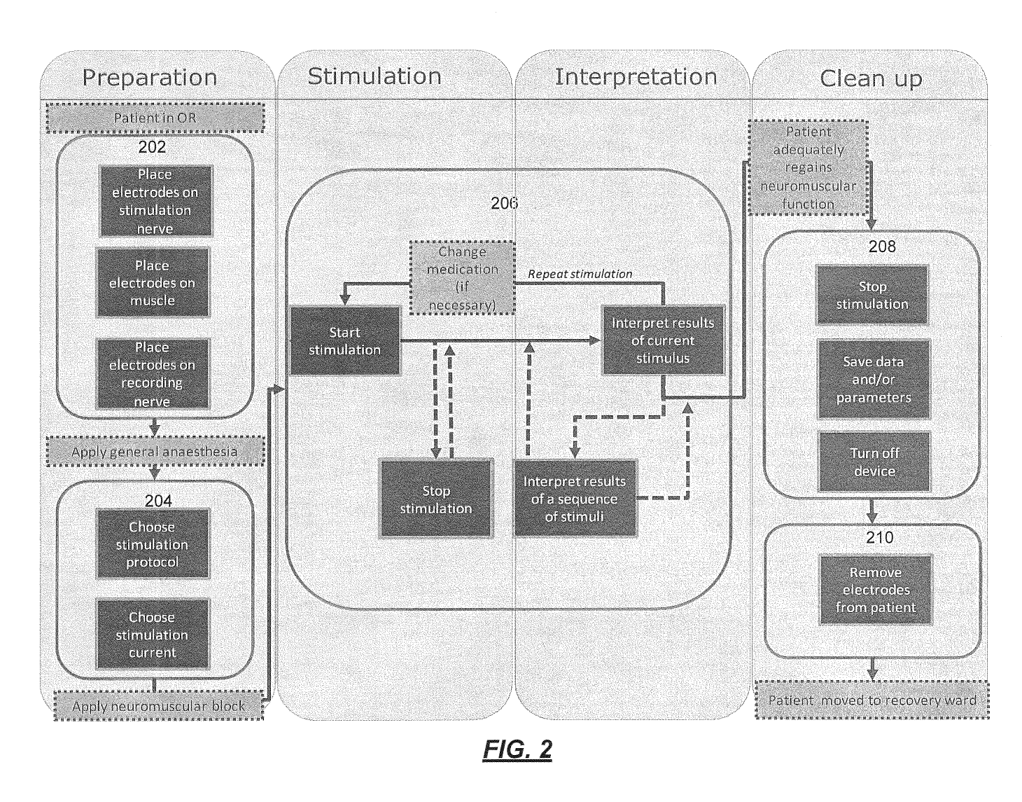

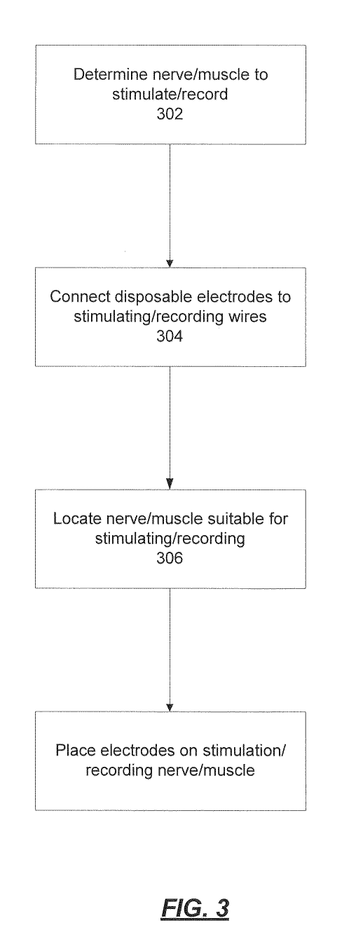

FIG. 2 illustrates a flow diagram of example operations performed within the system of FIG. 1. The example operations of FIG. 2 may be performed during surgery, for example. The example operations of FIG. 2 are divided into four phases, i.e., Preparation, Stimulation, Interpretation and Clean-up. During the preparation phase, the patient is admitted into the operating room. Then, at 202, the electrodes are placed on the patient. The electrodes may be, for example, the stimulation electrodes 102 and the sensor electrodes 104 and 106, as shown in FIG. 1. The process of placing the electrodes on the patient is discussed in detail with regard to FIG. 3.

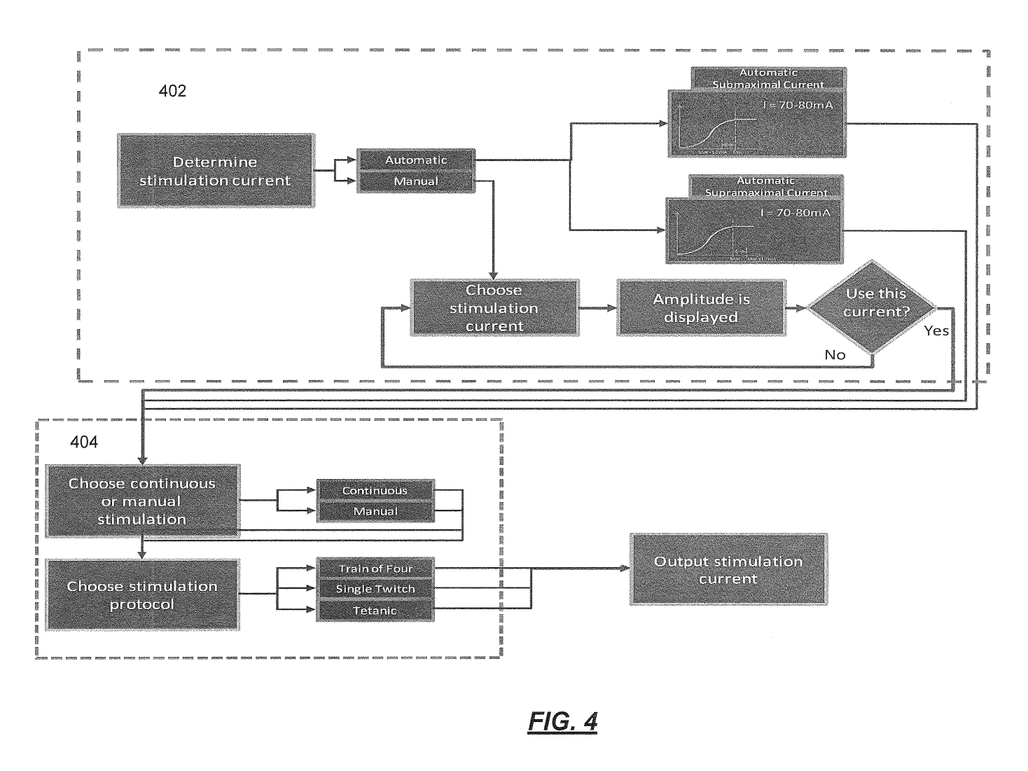

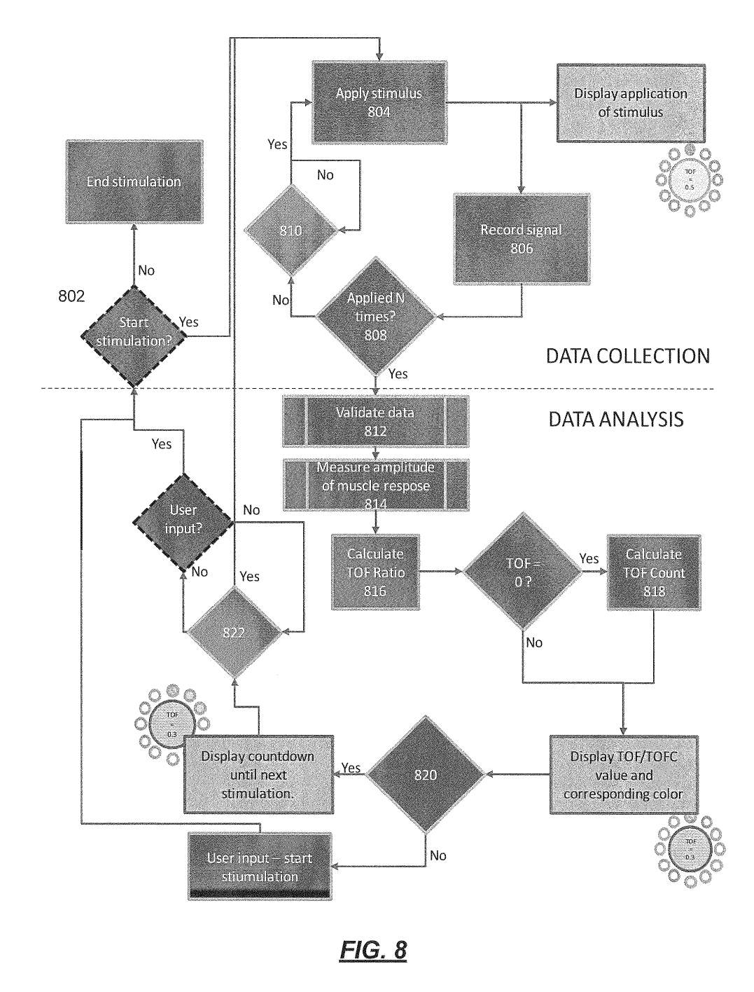

After placing the electrodes on the patient, general anesthesia may be administered to the patient. Next, at 204, the anesthesiologist may choose the stimulation current (i.e., stimulus intensity) and the stimulation protocol to be used while monitoring the neuromuscular block. Although, the term anesthesiologist is used throughout one skilled in the art will appreciate the system can be operated by other medical professionals or system operators and that the use of the term anesthesiologist does not limit the scope of the disclosed devices and methods. The anesthesiologist may choose the stimulation current either manually or automatically, which is discussed in detail with regard to FIG. 4. In addition, the anesthesiologist may choose from a number of stimulation protocols including but not limited to single twitch, TOF, tetanic and PTC. The process of choosing a stimulation protocol is discussed in detail with regard to FIG. 4, and the particular stimulation protocols are discussed in detail with regard to FIGS. 8-10.

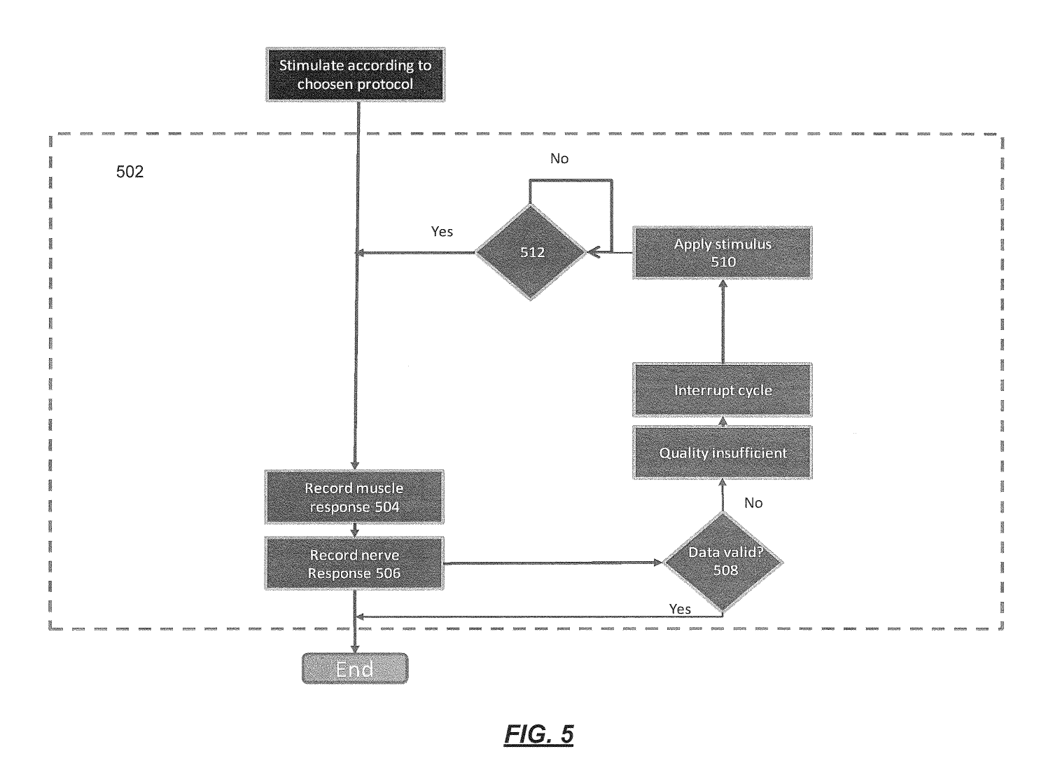

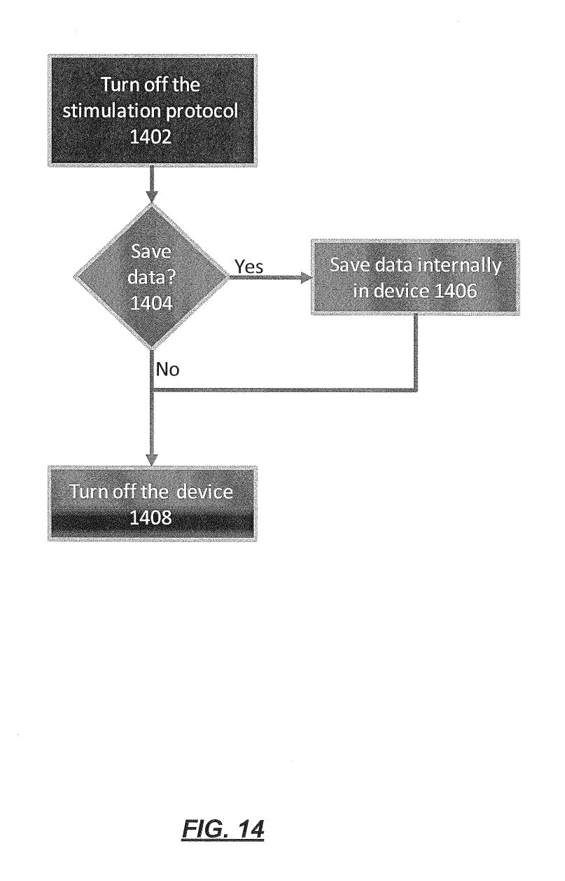

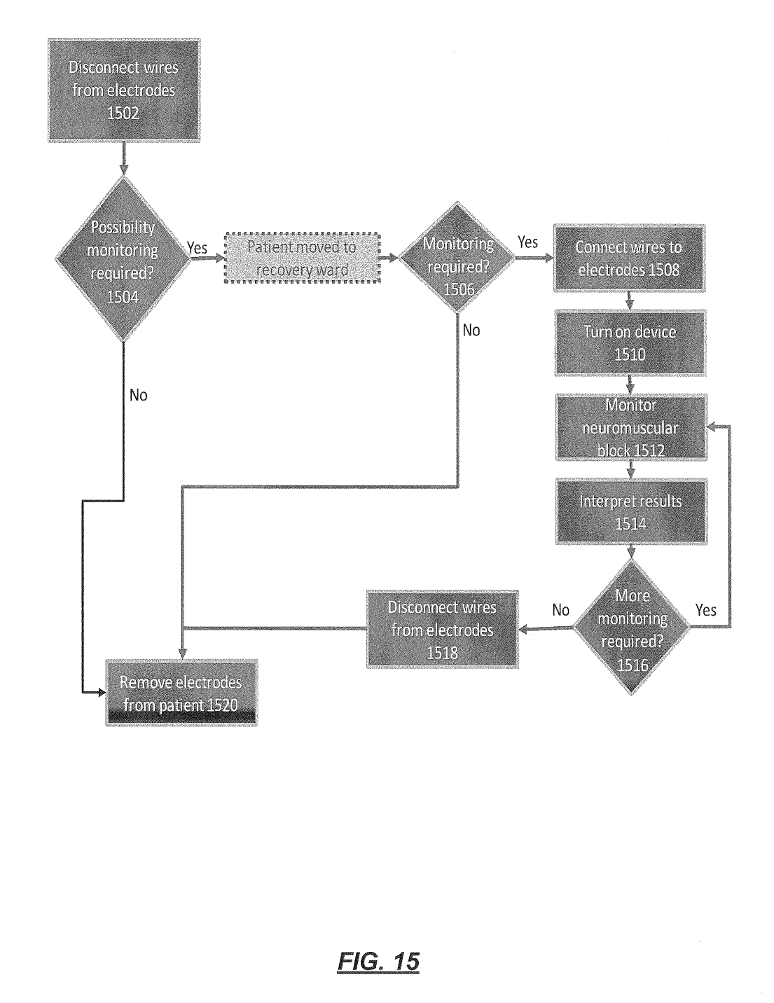

After choosing both the stimulation intensity and protocol, the anesthesiologist may begin to administer the muscle relaxant, which induces the neuromuscular block. In order to monitor anesthesia levels during the surgery, at 206, the anesthesiologist may monitor the neuromuscular block. The process of monitoring the neuromuscular block is discussed in detail with regard to FIGS. 5-11. Then, after the patient has adequately regained neuromuscular function at the conclusion of the surgery, the anesthesiologist may stop applying stimuli, save data and/or parameters, turn off the device and remove the electrodes from the patient at 208. This process is discussed in detail with regard to FIGS. 14 and 15.

FIG. 3 illustrates a flow diagram of example operations when placing electrodes. At 302, the anesthesiologist determines the nerves and muscles to stimulate and/or record. First, the anesthesiologist may choose the nerve to stimulate. For example, the anesthesiologist may choose to stimulate a motor nerve, which extends to the surface of a muscle where it contacts at the neuromuscular junction, such as the median or ulnar nerve at the wrist, the tibial nerve at the ankle, the facial nerve beneath the ear, etc. The evoked muscle responses may be recorded at the motor units innervated by the stimulated nerve. In addition, the evoked nerve responses may be recorded along the nerve pathway to either side of the stimulating site.

At 304, the anesthesiologist may connect the stimulating electrodes 102 and the sensor electrodes 104 and 106 to wires attached to the stimulating/recording unit 130. As discussed above, the stimulating and sensor electrodes 102, 104 and 106 may be attached to the stimulating/recording unit 130 through a custom connector.

At 306, the anesthesiologist may locate the nerves and muscles for stimulating and recording. As discussed above, the anesthesiologist may choose to stimulate a motor nerve such as the median or ulnar nerve at the wrist, the tibial nerve at the ankle, the facial nerve beneath the ear, etc. In order to record the evoked muscle responses, the anesthesiologist may locate the motor units that will be innervated by the stimulated nerve, such as the hand for the ulnar nerve, the foot for the tibial nerve or the eyebrow or jaw for the facial nerve. Then, the anesthesiologist may locate the nerve from which to record the evoked nerve response. The evoked nerve response may be recorded along the nerve pathway to either side of the stimulating site, but should preferably be recorded at least 5 cm away from the stimulating site to avoid interference. In one implementation, differential recording leads may be placed over active sites, i.e., one lead over the muscle and the other lead over the nerve to collect both responses in a single recording channel.

At 308, the anesthesiologist may place the stimulating electrodes 102 over the nerve to be stimulated and the sensing electrodes 104 and 106 over the motor units innervated by the stimulated nerve and along the nerve pathway, respectively.

FIG. 4 illustrates a flow diagram of example operations when determining a stimulation current and choosing a stimulation protocol. At 402, the anesthesiologist may determine the stimulation current (i.e., stimulation intensity) using, for example, the user-input controls of the control/visualization unit 132. The anesthesiologist may choose the stimulation intensity either manually or automatically. When choosing the stimulation intensity manually, the anesthesiologist may choose the actual stimulation current, for example, between 0-100 mA. In addition, the anesthesiologist may manually choose either the supra-maximal or submaximal current by increasing/decreasing the current in a predetermined sequence of incremental current changes in, for example 5 mA increments, until achieving maximal EMG+10% response (supramaximal) or threshold EMG+10% response (submaximal), respectively. Further, the anesthesiologist may choose the stimulation intensity automatically, which sets either the supra-maximal or submaximal current by applying the predetermined sequence of incremental current changes automatically until the maximal EMG+10% response or the threshold EMG+10% response is obtained. The system may also include a default stimulation intensity, i.e., supra-maximal, in the event that the anesthesiologist does not choose a stimulation intensity.

At 404, the anesthesiologist may choose the stimulation protocol. For example, the anesthesiologist may choose among single twitch, TOF, tetanic and PTC protocols using, for example, the user-input controls of the control/visualization unit 132. For the single twitch protocol, a single electrical pulse is applied, and the corresponding muscle response is recorded. For example, a single electrical pulse at supra-maximal intensity level may be applied for 200 μs, and the evoked muscle response may be recorded. The single electrical pulses of 200 or 300 μs duration may be repeated every 1 or every 10 seconds in a 1/sec (1 Hz) protocol or 1/10 sec (0.1 Hz) protocol.

For the TOF protocol, a predetermined pattern of stimuli may be applied at predetermined intervals. For example, a pattern of four electrical pulses each at supra-maximal intensity level for 200 μs or 300 μs may be applied every 500 ms. Each applied stimulus evokes a corresponding muscle response, which is recorded. Then, a ratio of the amplitude of a subsequent muscle response to the amplitude of a prior muscle response is calculated. For example, the ratio of the fourth muscle response to the first muscle response may be calculated (TOF ratio). In mild neuromuscular block, the evoked muscle responses progressively decrease in amplitude from the first to the fourth stimulus. Thus, by calculating the TOF ratio, it may be possible to determine the level of neuromuscular block because the TOF ratio corresponds to the level of neuromuscular block.

For the tetanic (TET) protocol, a predetermined pattern of stimuli may be applied at predetermined intervals similarly to the TOF protocol. However, in TET, a larger number of stimuli are applied at a higher frequency (i.e., 50 Hz, 70 Hz or 100 Hz) and for a longer total duration (i.e., 5 sec). The frequency used for the TET protocol may optionally be above a threshold frequency that achieves fusion of the muscular response to the stimulation, such as greater than 30 Hz in humans, for example. The frequency used for the TET protocol may be any frequency that achieves fusion of the muscular response to the stimulation, which may be a frequency above 30 Hz. For example, a pattern of 250 or 500 electrical pulses (each of 200 μsec duration), and each at supra-maximal (or less than supramaximal) intensity level at a rate of 50 or 100 Hz for five seconds may be applied. During normal neuromuscular transmission, the evoked muscle responses to the tetanic stimulation fuse into a single sustained contraction of the muscle. In other words, the normal (unblocked) muscle response to the tetanic stimulation is sustained (fused) for the duration of the stimulation. However, during a non-depolarizing neuromuscular block, the response to the tetanic stimulation will not be sustained (i.e., fade occurs). Thus, it may be possible to determine the level of neuromuscular block by calculating the ratio of the amplitude of the muscle response at the end of the stimulation to the amplitude of the muscle response at the beginning of the stimulation. Alternatively, because there may be some amplitude variation in the evoked muscle responses at the beginning of the tetanic stimulation, a ratio of the amplitude of any response toward the end of the stimulation to the amplitude of any response toward the beginning of the stimulation may be calculated, and a value less than 1.0 demonstrates the presence of neuromuscular block. For example, there may be some amplitude variation in the evoked responses during the first 1-3 seconds of the stimulation. In some implementations, the response towards the beginning of the stimulation with the largest amplitude may be used in the ratio.

For the PTC protocol, a tetanic stimulation may be applied as discussed above. After the end of the tetanic stimulation, single twitch stimuli may be applied at predetermined intervals. For example, single-twitch stimuli may be applied at a rate of 1 Hz beginning 30 seconds after the end of the tetanic stimulation, and the number of responses to the single-twitch stimuli may be counted. The tetanic stimulation causes release of all available neurotransmitter from the nerve terminal, which may restore twitch response for a short interval following the tetanic stimulation. During deep neuromuscular block, the time until return of the first response to TOF stimulation is related to the number of PTC twitch responses present at a given time.

In addition to choosing a stimulation protocol, the anesthesiologist may also choose whether the stimulation will be continuous or manual. For manual stimulation, the anesthesiologist may trigger application of the stimuli using the user-input controls of the control/visualization unit 132. For continuous stimulation, successive stimulation protocols may be applied at predetermined time intervals. Single twitch, TOF, tetanic and PTC protocols may be repeated every 1 second, 12 seconds, 120 seconds and 120 seconds, respectively, for example.

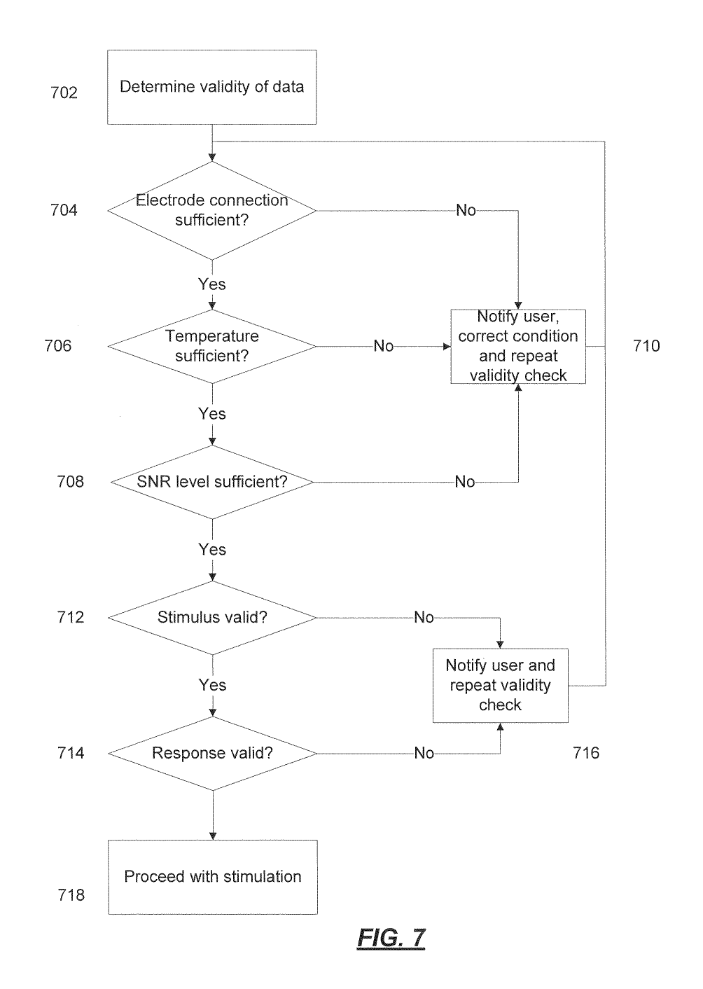

FIG. 5 illustrates a flow diagram of example operations when monitoring neuromuscular block. At 502, the anesthesiologist may apply stimuli according to the chosen protocol. At 504, the muscle response is sensed and recorded using the sensing electrodes 104, and at 506, the nerve response is sensed and recorded using the sensing electrodes 106. At 508, a determination is made as to whether the collected data are valid. This is discussed in detail with regard to FIG. 7. At 510, a subsequent stimulus may be applied after a predetermined period of time has elapsed since the previous stimulus, which is determined at 512.

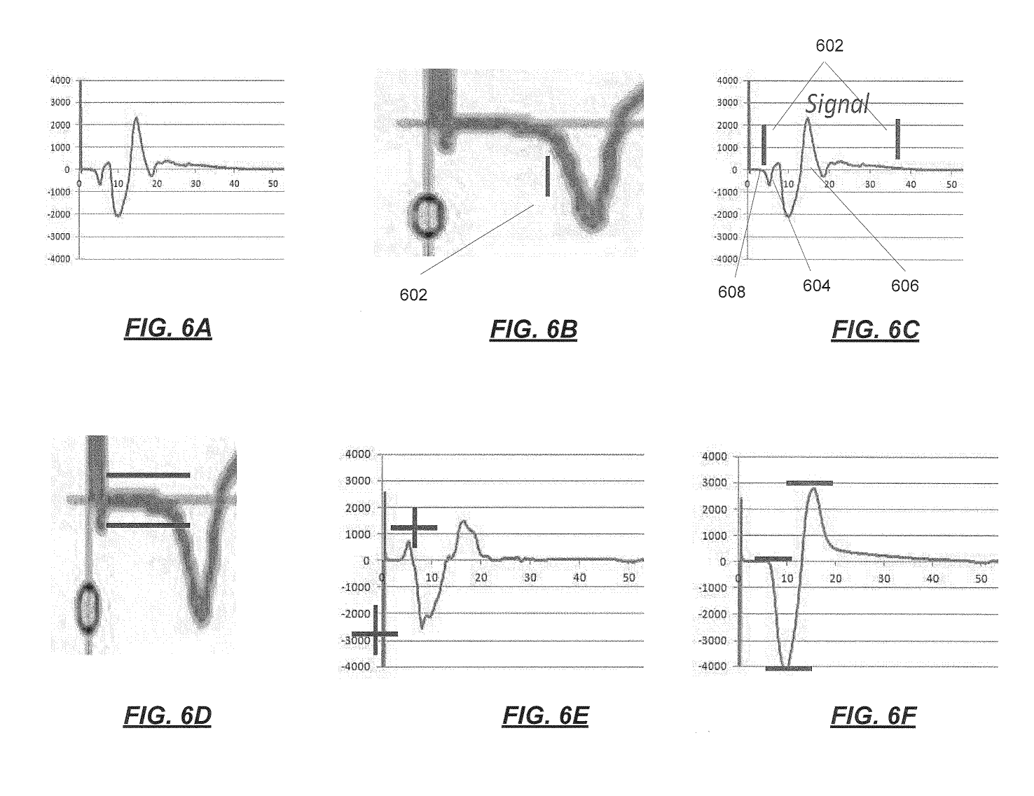

FIGS. 6A-6F illustrate data collected in response to a nerve stimulus. In order to measure the nerve and muscle responses, the systems and methods disclosed herein begin with an epoch of collected data in response to a stimulus. For example, FIG. 6A illustrates data collected (i.e., a detected voltage signal) by the sensors 104 and 106 in response to the applied stimulus. The collected data include noise (the stimulus artifact), the electrical activity of the nerve (i.e., the nerve response) and the electrical activity of the muscle (i.e., the muscle response). In one implementation, the noise, nerve response and muscle response may be separated from one another before each is measured.

First, the limits of the neuromuscular activity may be detected by identifying the point where the detected voltage signal deviates significantly from the baseline value [i.e., the background (pre-stimulus or immediately post-response) level of electrical activity (characterized by, for example, the Root Mean Square (RMS) amplitude)], for example, by working inward from the high- and low-ends of the sequence of values making up the detected voltage signal. The baseline level of electrical noise is the background noise and may include all ambient, intrinsic and extrinsic noise. Then, when both the slope and amplitude differ by a predetermined amount from their respective baseline values, a limit may be declared and a fiducial mark 602 may be placed to indicate the location. The point where both the slope and amplitude differ by a predetermined amount from their respective baseline values may be visually identified by a “knee” in the detected voltage signal. The “knee” and fiducial mark 602 are shown in FIG. 6B, which illustrates a portion of FIG. 6A. The portions of the detected voltage signal that precede and follow the fiducial marks 602 as shown in FIG. 6C are noise-only segments that can be assessed for artifact and unacceptable levels of interference. The portion of the detected voltage signal between the fiducial marks 602 is the neuromuscular response, which contains both the nerve and muscle responses and noise.

The region of the detected voltage signal shown in FIG. 6C and marked “Signal” may be further subdivided into the respective nerve and muscle responses contained in the detected voltage signal by a number of means. First, the muscle response 606 generally occurs later in time (and is of higher amplitude) than the nerve response 604 because the muscle activates after the nerve. Thus, it may be possible to identify and then subtract the muscle response 606 from the detected voltage signal to obtain the nerve response 604. Second, the constant portions of the detected voltage signal are presumed to belong to the nerve response 604 because the muscle response 606 is more variable than the nerve response 604, which is constant over time when present, and therefore, the nerve response 604 can be identified and then subtracted from the detected voltage signal to obtain the muscle response 606. Third, the muscle response 606 has a characteristic shape that may be fitted to and then subtracted from the detected voltage signal to obtain the nerve response 604. The noise 608, nerve response 604 and the muscle response 606 are shown separately in FIGS. 6D, 6E and 6F, respectively.

After the three portions of the detected voltage signal (i.e., noise 608, nerve response 604 and muscle response 606) have been separated, each respective portion may be measured. The noise 608 may be analyzed to determine whether artifact is present. Additionally or alternatively, the noise 608 may be statistically analyzed, the slope and/or RMS value across the region may be calculated or the frequency content may be estimated by counting zero crossings. The nerve response 604 may be assessed for consistency, for example by determining whether the nerve response 604 changes shape or amplitude. The nerve response 604 is expected to be consistent, or not changing in shape or amplitude. The nerve response 604 may also be assessed by analyzing the amplitude and intervals between major features, or performing correlation checks of the aligned detected voltage signal and a template composed from prior recordings. The muscle response 606 may be assessed by measuring the peak-to-peak amplitude, the baseline-to-peak amplitude or the difference between peak values.

FIG. 7 illustrates a flow diagram of example operations when determining validity of collected data. Prior to analyzing the collected data, a determination is made as to whether the collected data are valid. The validity determination begins at 702. For example, electrode connection integrity, temperature, noise levels, etc. may be analyzed. At 704, a determination is made as to whether the electrode connection is sufficient. For example, the electrode connection may be sufficient if the impedance is between 500 and 5,000 Ohms. At 706, a determination is made as to whether the temperature is sufficient. When temperature is insufficient (i.e., too low), nerve function may be impaired by the low temperature. Thus, low temperature may be detected, and the area may be heated to normal body temperature in order to eliminate the possibility of errors. In some implementations, temperature of the skin may be detected in order to estimate temperature of the nerve because the temperatures will be approximately the same at any given time. For example, the temperature may be sufficient if it is greater than or equal to 34° C. At 708, a determination is made as to whether the signal-to-noise ratio (SNR) is sufficient. In one implementation, the electrode connection, temperature and signal-to-noise ratio must all be sufficient to proceed. In other implementations, the above condition may not be required. If conditions are insufficient, then the user may be notified at 710 so that the insufficient condition may be remedied, and the validity checks may be repeated. As discussed above, the insufficient condition may be displayed on the control/visualization unit 132.

At 712, a determination is made as to whether the stimulus was delivered. When the stimulus is moved off of (i.e., distant from) the motor nerve and the stimulus is thus unable to trigger a nerve response, both the muscle response and the nerve response, as sensed by the sensing electrodes 104 and 106, will be non-existent. The nerve response should be present even during full neuromuscular block. Thus, if the nerve response is non-existent, then the stimulus was not delivered (or the stimulus was not sufficient to trigger a nerve response). In addition, it may be possible to detect whether the stimulus was delivered by detecting some twitch based on motion artifacts appearing in the impedance channel. If the stimulus was not delivered, the user may be notified, and the validity checks may be repeated at 716. As discussed above, the insufficient condition may be displayed on the control/visualization unit 132.

At 714, a determination is made as to whether the response is valid. For example, the nerve and muscle responses may be analyzed to determine whether each response is present, the amplitudes are decreasing, the response latency is consistent, etc. If the response is invalid, the user may be notified, and the validity checks may be repeated at 716. As discussed above, the insufficient condition may be displayed on the control/visualization unit 132. After determining that the collected data are valid, it is possible to proceed with the stimulation at 718 and measure the amplitude of the muscle response.

FIG. 8 illustrates a flow diagram of example operations when applying the TOF protocol. The TOF protocol consists of applying a predetermined pattern of stimuli at predetermined intervals to the motor nerve. At 802, the TOF protocol begins, and at 804 the first stimulus is applied. For example, the stimulus may be a 200 μs or 300 μs, square-wave, monophasic, fixed width between 100 μs and 300 μs constant current electrical pulse. Optionally, the stimulus duration may be longer or shorter than 200 μs, including but not limited to a duration between 100 and 300 μs. As discussed above, the control/visualization unit 132 may indicate that the stimulus has been applied, for example using an indicating light. At 806, the nerve and muscle responses are recorded by the sensing electrodes 104 and 106. Thereafter, at 808, a determination is made as to whether a predetermined number of stimuli have been applied. In one implementation, the predetermined number is preferably four stimuli, but it may also be five, six, seven, etc. If Yes, at 812, a determination is made as to whether the collected data are valid, which is discussed in detail with regard to FIG. 7. If No, a subsequent stimulus is applied after a predetermined time interval, for example 500 ms, has elapsed. However, the predetermined time interval may be greater or less than 500 ms.

At 814, the amplitude of the muscle response is measured. As discussed above, the amplitude may be the peak-to-peak or the baseline-to-peak amplitude. The measured amplitude may be compared to a control amplitude to determine the level of neuromuscular block. For example, the control amplitude may be zero. When the predetermined pattern of stimuli is applied to the patient before administration of the muscle relaxants, the amplitude of the muscle responses are expected to be approximately equal and non-zero. However, as muscle relaxants are administered to the patient, the amplitude of each subsequent muscle response diminishes. In one implementation, the amplitude decreases to zero, preferably by the fourth recorded muscle response, which may indicate a certain degree of neuromuscular block.

At 816, the TOF ratio may be determined by calculating a ratio of amplitudes of any two, distinct muscle responses to a train of sequentially applied stimuli. In some implementations, the ratio may be a ratio of the amplitude of a subsequent muscle response (i.e., recorded later in time) to the amplitude of a previous muscle response (i.e., recorded earlier in time). For example, the train-of-four ratio is the ratio of the amplitude of the fourth sequentially applied stimulus to the first sequentially applied stimulus in a train of sequentially applied stimuli. The TOF ratio may then be compared to a control ratio (which should preferably be 1.0). Preferably, the TOF ratio will be a ratio of the amplitude of the fourth muscle response to the amplitude of the first muscle response, but can alternatively be the ratio of the amplitudes of any of the first, second, third, fourth, fifth, six, etc. muscle responses. In an unblocked state, the TOF ratio is approximately 1.0. As the neuromuscular block deepens, the TOF ratio falls progressively to 0.0. Thus, a smaller TOF ratio, i.e., one that approaches 0.0, corresponds to a greater level of neuromuscular block, and a TOF ratio of the fourth to the first muscle response of 0.0 indicates approximately greater than or equal to 80% neuromuscular block (receptor occupancy).

Next, a determination is made as to whether the TOF ratio equals zero. If No, the control/visualization unit 132 may display the TOF ratio value, as well as a corresponding color. The display may utilize different colors to indicate different levels of neuromuscular block. For example, the color green may be used to represent a TOF ratio between 1.0 and 0.90, the color yellow may be used to represent a TOF ratio between 0.89 and 0.40 and the color red may be used to represent a TOF ratio between 0.39 and 0.01. If Yes, at 818, the TOF count is calculated. For example, when the TOF ratio is 0.0 (i.e., the fourth muscle response is non-existent), a determination is made as to how many stimuli (i.e., first, second and third stimuli) exhibited a non-zero response. As neuromuscular block deepens, the TOF count decreases from three counts to zero. For example, when the TOF ratio is 0.0 and the TOF count is zero, the neuromuscular block is approximately greater than or equal to 95%. In contrast, as neuromuscular block lessens, the TOF count increases. When the TOF ratio is 0.9 (and the TOF count is, by definition, four), the neuromuscular block is approximately less than or equal to 70%. This level of neuromuscular function (less than 70% block) is considered the threshold for adequate recovery. The TOF count value may then be displayed on the control/visualization unit 132, along with a corresponding color. In other implementations, the TOF count may be calculated for greater than four applied stimuli.

At 820, a determination is made as to whether the stimulation mode is manual or continuous. When the stimulation mode is manual, a subsequent stimulation protocol is applied only after the user triggers application using for example, user-input controls of the control/visualization unit 132. When the stimulation mode is continuous, at 822, a determination is made as to whether a predetermined time between stimulation protocols has elapsed. Preferably, the predetermined sequential time for the TOF protocol is 12 seconds. In addition, the control/visualization unit 132 may display the time remaining until application of the next stimulation protocol.

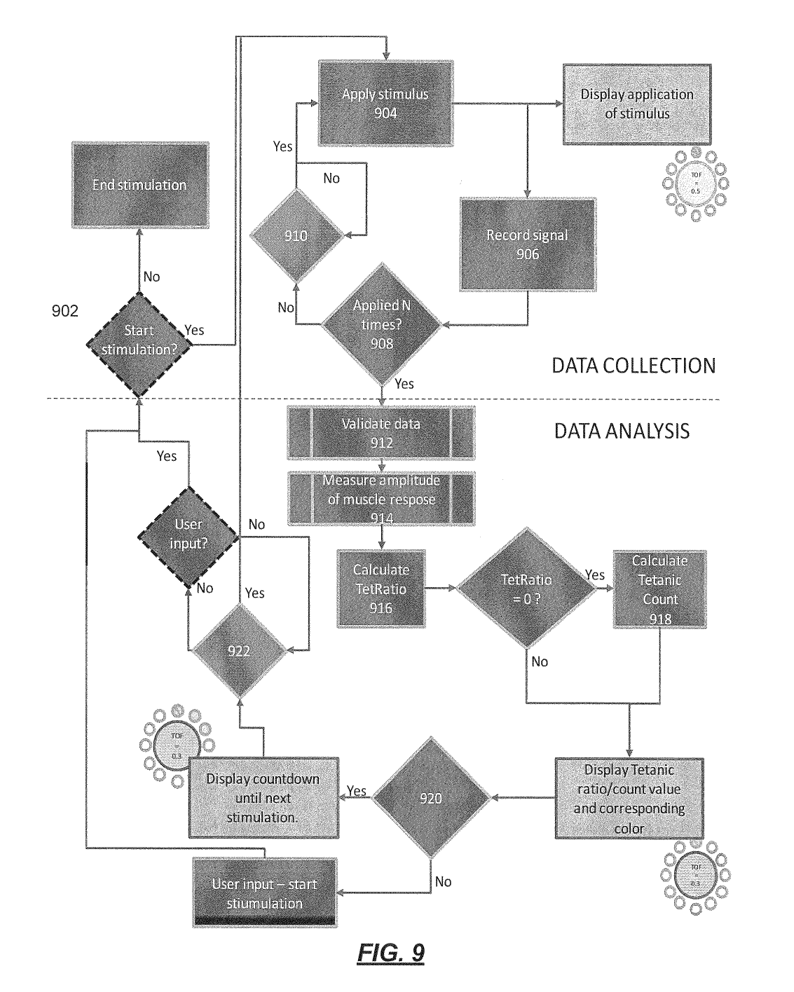

FIG. 9 illustrates a flow diagram of example operations when applying the tetanic test protocol. Similarly to the TOF protocol, the tetanic (TET) protocol consists of a predetermined pattern of stimuli applied at predetermined intervals. Unlike the TOF protocol, however, the tetanic protocol consists of applying a larger number of stimuli at a higher frequency. At 902, application of the stimuli begins, and at 904, the first stimulus is applied. For example, 250 or 500 electrical pulses may be applied at a rate of 50 or 100 Hz in a five-second period. In addition, each stimulus (electrical pulse) may have a duration of 200 μs, or, optionally, a duration greater than or less than 200 μs. Application of the stimulus may be displayed at the control/visualization unit 132 using an indicating light and/or a sound, for example. At 906, the nerve and muscle responses are recorded by the sensing electrodes 104 and 106. Thereafter, at 908, a determination is made as to whether a predetermined number of stimuli (i.e., 250 or 500) have been applied. If Yes, at 912, a determination is made as to whether the collected data are valid, which is discussed in detail with regard to FIG. 7. If No, a subsequent stimulus is applied after a predetermined time interval, for example 4 ms or 2 ms, when the pulses are applied at a rate of 50 or 100 Hz, respectively, has elapsed at 910.

At 914, the amplitude of the muscle responses is measured, and at 916, the tetanic ratio is calculated. Similarly to the TOF ratio, the tetanic ratio may be the ratio of an amplitude of a subsequently applied stimulus (or series of stimuli) to an amplitude of a previously applied stimulus (or series of stimuli), i.e., the last stimulus to the first stimulus in the train of stimuli (or a combination of later-in-time series of stimuli to earlier-in-time series of stimuli). However, as discussed above, the ratio may be the ratio of amplitudes of any two, distinct muscle responses to a train of sequentially applied stimuli. As the neuromuscular block deepens, the tetanic ratio falls progressively from a normal baseline of 1.0 towards 0.0. Thus, a smaller tetanic ratio, i.e., one that approaches 0.0, corresponds to a greater level of neuromuscular block. If the tetanic ratio equals zero, at 918, the tetanic duration may be calculated. The tetanic duration may be calculated by estimating the duration of the time interval between the non-zero start and the end of the response, i.e., 0.1-4.9 seconds. As discussed above, during normal neuromuscular transmission, the evoked muscle responses to the tetanic stimulation merge into a single sustained contraction of the muscle. However, during neuromuscular block, the amplitude of responses to the tetanic stimulation will not be sustained (i.e., fade occurs). Accordingly, the level of neuromuscular block may correspond to the time interval of the response. In addition, the tetanic duration value may be displayed by the control/visualization unit 132, along with the corresponding color.

At 920, a determination is made as to whether the stimulation mode is manual or continuous. When the stimulation mode is manual, a subsequent stimulation protocol is applied only after the user triggers application using for example, user-input controls of the control/visualization unit 132. When the stimulation mode is continuous, at 922, a determination is made as to whether a predetermined time between stimulation protocols has elapsed. Preferably, the predetermined time for tetanic protocol is 120 seconds (i.e., the duration elapsed between successive tetanic stimulations is at least 120 sec in order to avoid the phenomenon of “post-tetanic potentiation” which would invalidate the neuromuscular responses). In addition, the control/visualization unit 132 may display the time interval until application of the next stimulation protocol.

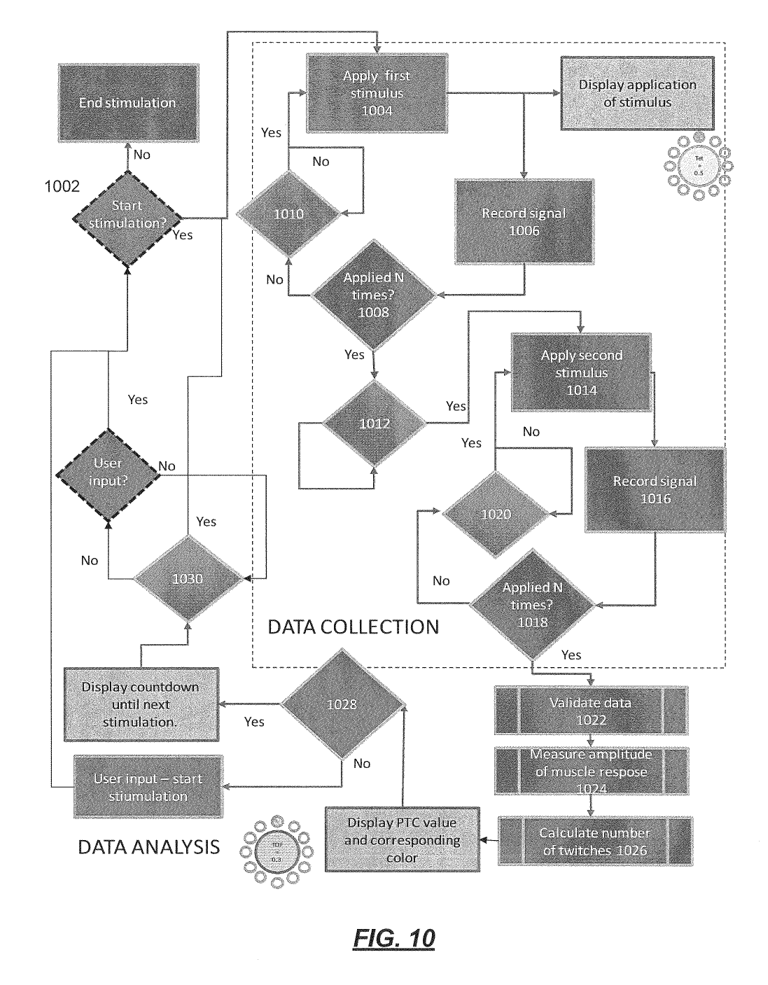

FIG. 10 illustrates a flow diagram of example operations when applying the post-tetanic count (PTC) test protocol. When a deep neuromuscular block is achieved, and estimation using either the TOF protocol or the tetanic protocol is not elicited, it may be possible to elicit a response using a special stimulus protocol, i.e., the PTC protocol. At 1002, the stimulation protocol begins, and at 1004, the first stimulus is applied. Preferably, the first stimulus is a tetanic stimulation, or a pattern of 250 or 500 stimuli (each of 200 μs duration) applied at, optionally, 50 or 100 Hz during a five-second period. Optionally, the duration of each stimulus may be longer or shorter than 200 μs. At 1006, the nerve and muscle responses are recorded using the sensor electrodes 104 and 106. At 1008, a determination is made as to whether a predetermined number of stimuli have been applied, i.e., 250 or 500. If No, a subsequent stimulus is applied after a predetermined time interval, for example 4 ms or 2 ms, when the pulses are applied at a rate of 50 or 100 Hz, respectively, has elapsed at 1010. If Yes, after the first stimulus is complete, a determination is made as to whether a predetermined time interval has elapsed at 1012. For example, in one implementation, the predetermined time interval is 30 seconds. After the predetermined time interval has elapsed, at 1014, a second stimulus is applied. For example, the second stimulus may be a single twitch. At 1016, the nerve and muscle responses are recorded using the sensor electrodes 104 and 106. At 1018, a determination is made as to whether the second stimulus has been applied a predetermined number of times, i.e., 20, at a frequency of 1 Hz (1 stimulation/sec).

At 1022, after the second stimulus is complete, a determination is made as to whether the collected data are valid, which is discussed in detail with regard to FIG. 7. At 1024, the amplitudes of the muscle responses are measured. At 1026, the number of second stimuli (delivered at a frequency of 1 Hz) that elicit a non-zero response are counted. As the neuromuscular block deepens, the number of second stimuli that elicit a response decreases. In other words, the PTC value decreases for deeper levels of neuromuscular block.

At 1028, a determination is made as to whether the stimulation mode is manual or continuous. When the stimulation mode is manual, a subsequent stimulation protocol is applied only after the user triggers application using for example, user-input controls of the control/visualization unit 132. When the stimulation mode is continuous, at 1030, a determination is made as to whether a predetermined time between stimulation protocols has elapsed. Preferably, the predetermined time for the PTC protocol is 120 seconds. In addition, the control/visualization unit 132 may display the time interval until application of the next stimulation protocol.

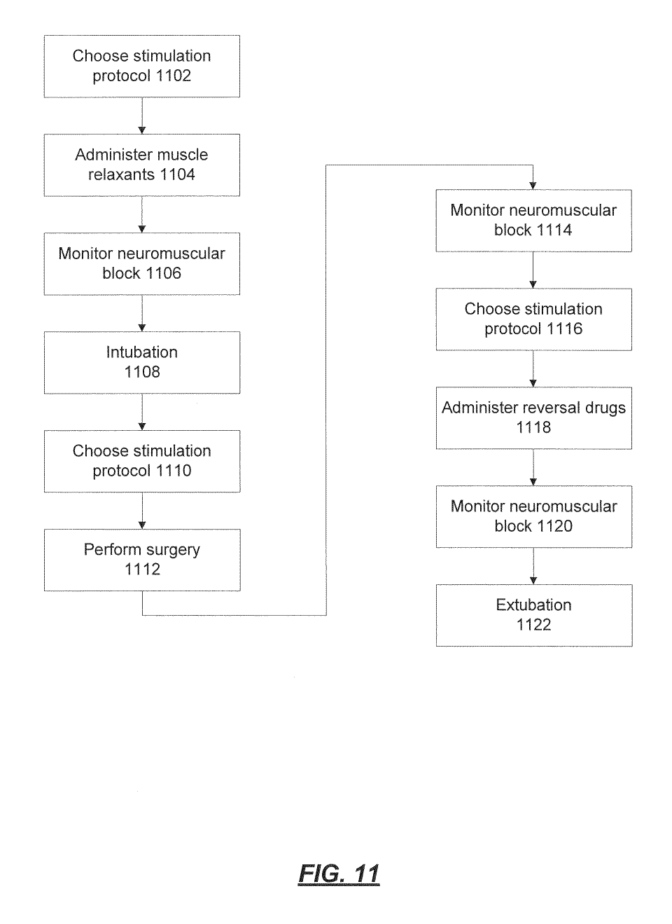

FIG. 11 illustrates a flow diagram of example operations when monitoring neuromuscular block during surgery. At 1102, the anesthesiologist may choose a stimulation protocol such as the single twitch, the TOF, the tetanic or the PTC test protocol for use at the beginning of the surgery. Next, at 1104, the anesthesiologist may administer the muscle relaxants in order to induce the neuromuscular block. After administering the muscle relaxants, the anesthesiologist may monitor the neuromuscular block at 1106. For example, in one implementation, the anesthesiologist may monitor the neuromuscular block prior to intubation using the TOF protocol, and when the TOF ratio drops to zero and remains at zero for at least three consecutive readings, the neuromuscular block may be maximal. At this point, at 1108, the patient's trachea may be intubated.

At 1110, the anesthesiologist may again choose a stimulation protocol while reducing the dose of the muscle relaxants prior to performance of the surgery at 1112. For example, the anesthesiologist may monitor the neuromuscular block using the TOF protocol until a minimal, non-zero TOF ratio is obtained. During the surgery, the anesthesiologist may continue to monitor the neuromuscular block at 1114. At the conclusion of the surgery, the anesthesiologist may again choose a stimulation protocol at 1116 prior to administering reversal drugs, i.e., antagonists at 1118. After administering antagonists, the anesthesiologist may monitor the neuromuscular block at 1120. For example, the anesthesiologist may monitor the neuromuscular block using the TOF protocol until twitch returns and the TOF ratio normalizes to at least 0.90, and preferably 1.0. Finally, at 1122, the breathing tube may be removed from the patient's trachea.

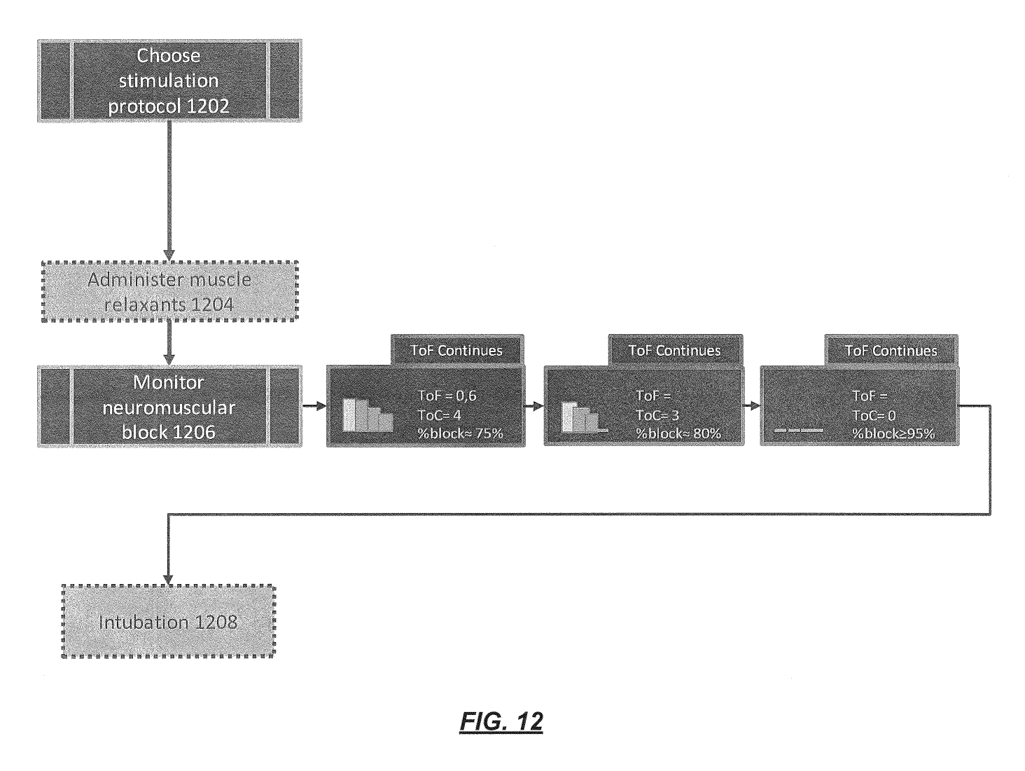

FIG. 12 illustrates example operations of performing the TOF test protocol prior to intubation. At 1202, the anesthesiologist may choose the TOF test protocol in the continuous mode. Then, at 1204, the anesthesiologist may administer the muscle relaxants in order to induce the neuromuscular block. At 1206, the anesthesiologist begins to monitor the neuromuscular block. During monitoring, the control/visualization unit 132 may display a bar graph indicating the amplitude of the muscle responses, the ratio, the count, the percentage block, etc. In addition, the response to each successive stimulus application may be scrolled on control/visualization unit 132, for example. At 1208, the patient's trachea may be intubated.

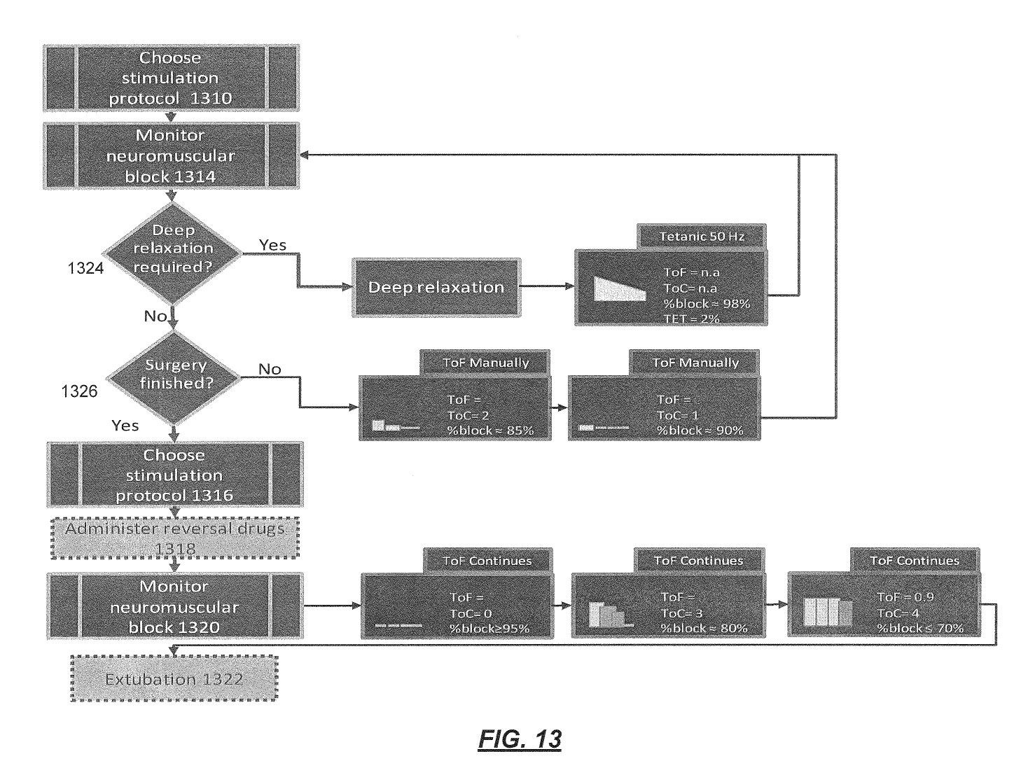

FIG. 13 illustrates example operations when performing the tetanic test protocol (TET) during surgery and the TOF test protocol after administering reversal drugs. At 1310, the anesthesiologist may choose the stimulation protocol to be used for monitoring the neuromuscular block during surgery at 1314. For example, when deep relaxation is required at 1324, the anesthesiologist may monitor the neuromuscular block using the tetanic protocol in the continuous mode. When deep relaxation is not required, but the surgery is not complete at 1326, the anesthesiologist may choose to monitor the neuromuscular block using the TOF protocol in the manual mode. At the conclusion of the surgery, the anesthesiologist may again choose a stimulation protocol at 1316 prior to administering the antagonists at 1318. After administering the antagonists, the anesthesiologist may choose to monitor neuromuscular block at 1320 using the TOF protocol in the continuous mode. During monitoring, the control/visualization unit 132 may display a bar graph indicating the amplitude of the muscle responses, the ratio (in appropriate color), the count, the percentage block, etc. In addition, the response to each successive stimulus application may be scrolled on control/visualization unit 132, for example. When twitch returns and the TOF ratio normalizes to at least 0.90, and preferably 1.0, the breathing tube may be removed from the patient's trachea at 1322.

FIG. 14 illustrates example operations when turning off the monitoring device. At 1402, the anesthesiologist may turn off the stimulation protocol. Then, at 1404, a determination is made as to whether the data are to be saved. If Yes, the collected data may be saved internally in the device at 1406. As discussed above, it may be possible to download the collected data from the control/visualization unit 132 using the external communication link 124. After the collected data have been saved or it is determined that it is not necessary to save the collected data, the anesthesiologist may turn the device off at 1408. In some implementations, the data may be interfaced with an electronic medical record storage system for storing in the patient's electronic medical record.

FIG. 15 illustrates example operations when removing the electrodes from the patient. At 1502, the anesthesiologist may disconnect the stimulation and sensing electrodes 102, 104 and 106 from the stimulating/recording unit 130. As discussed above, the electrodes 102, 104 and 106 may be connected to the wires using a custom key. At 1504, a determination is made as to whether additional monitoring will be required. If No, the anesthesiologist may remove the electrodes 102, 104 and 106 from the patient at 1520. If Yes, the patient may be moved to the recovery ward without removing the electrodes 102, 104 and 106.

After moving the patient to the recovery ward, a determination is made as to whether additional monitoring will be required at 1506. If No, the anesthesiologist may remove the electrodes 102, 104 and 106 from the patient at 1520. If Yes, at 1508, the anesthesiologist may connect the electrodes 102, 104 and 106 stimulating/recording unit 130. Then, at 1510, the anesthesiologist may turn on the device and begin monitoring the neuromuscular block at 1512. After interpreting the results at 1514, a determination is made as to whether additional monitoring is required at 1516. If Yes, the anesthesiologist may continue to monitor the neuromuscular block at 1512. If No, at 1518, the anesthesiologist may disconnect the electrodes 102, 104 and 106 from the stimulating/recording unit 130, and then remove the electrodes 102, 104 and 106 from the patient at 1520.

With reference to FIG. 16, an example system for implementing aspects described herein includes a processing device 1620. In its most basic configuration, the processing device 1620 typically includes at least one processing unit 1602 and memory 1604. Depending on the exact configuration and type of processing device, memory 1604 may be volatile (such as random access memory (RAM)), non-volatile (such as read-only memory (ROM), flash memory, etc.), or some combination of the two.

The processing device 1620 typically includes a variety of computer readable media. The computer readable media can be any available media that can be accessed by processing device 1620 and includes both volatile and non-volatile media. The computer readable media may be stored on volatile or non-volatile memory, and the memory can be implemented in any method or technology for storage of information such as computer readable instructions, data structures, program modules or other data. Memory includes, but is not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by the processing device 1620.

It should be understood that the various techniques described herein may be implemented in connection with hardware or software or, where appropriate, with a combination of both. Thus, the methods and systems of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a processor, the processor becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations

A number of embodiments of the invention have been described. Nevertheless, it will be understood that various modifications may be made without departing from the spirit and scope of the invention. Accordingly, other embodiments are within the scope of the following claims.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a method is disclosed and discussed each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the materials for which they are cited are hereby specifically incorporated by reference in their entireties.

1. A method for assessing neuromuscular blockade in a subject having been administered a muscle relaxant agent, comprising:
a. stimulating a motor nerve to cause an evoked muscle response;
b. recording the evoked muscle response;
c. identifying a peak of the recorded evoked muscle response; and
d. determining the amplitude of the peak from baseline, wherein the determined amplitude compared to a control amplitude indicates a level of neuromuscular blockade in the subject.

2. The method of claim 1, further comprising identifying the start of the evoked muscle response and using the level of muscle electrical activity prior to the start for the baseline.

3. The method of claim 1, wherein the control amplitude is the amplitude determined from the baseline to peak of a prior or subsequent evoked muscle response caused by stimulating the same motor nerve.

4. The method of claim 3, wherein the prior or subsequent evoked muscle response is produced during a repeated train of stimuli to the motor nerve.

5. The method of claim 4, wherein the repeated train of stimuli includes four stimuli, each stimulus capable to cause an evoked muscle response.

6. The method of claim 5, wherein one or more of the four stimuli do not produce a corresponding evoked muscle response.

7. The method of claims 5, wherein the train of stimuli produces a plurality of evoked muscle responses.

8. The method of claim 7, further comprising identifying a peak of at least one of the plurality of evoked muscle responses.

9. The method of claim 5, wherein the determined amplitude is the amplitude determined from the baseline to peak of the evoked muscle response caused by the fourth stimulation.

10. The method of claim 5, wherein the control amplitude is the amplitude determined from the baseline to peak of the evoked muscle response caused by the first, second or third stimulation.

11. The method of claim 9, wherein the determined amplitude has a value of zero.

12. The method of claim 9, wherein the comparison of the determined amplitude corresponding to the first stimulus and the amplitude corresponding to the fourth stimulus includes determining a ratio of the amplitude corresponding to the fourth stimulus and the amplitude corresponding to the first stimulus.

13. The method of claim 12, wherein the determined amplitude has a value of zero and the ratio is zero.

14. The method of claim 13, further comprising determining if there was an evoked muscle response corresponding to the first, second and third stimuli.

15. The method of claim 14, further comprising displaying a count number representing the count of the number of evoked muscle responses corresponding to the number of first, second and third stimuli that elicit non-zero amplitude responses.

16. The method of claim 14, further comprising stimulating the motor nerve in a tetanic protocol if there is no evoked muscle response corresponding to the first, second and third stimuli.

17. The method of claim 4, wherein the repeated train of stimuli is delivered during a tetanic stimulation protocol of the motor nerve.

18. The method of claim 1, wherein each peak identified is the peak having the largest negative value in its corresponding evoked muscle response.

19. The method of claim 1, wherein each peak identified is the peak having the largest positive value in its corresponding evoked muscle response.

20. A method for assessing neuromuscular blockade in a subject having been administered a muscle relaxant agent, comprising:
a. stimulating a motor nerve to cause an evoked muscle response;
b. recording the evoked muscle response;
c. identifying a first peak of the recorded evoked muscle response;
d. identifying a subsequent peak of opposite polarity of the recorded evoked muscle response; and
e. determining the peak-to-peak amplitude by identifying the difference between the first and subsequent peak, wherein the peak-to-peak amplitude compared to a control amplitude indicates a level of neuromuscular blockade in the subject.

21. The method of claim 20, wherein the control amplitude is the peak-to-peak amplitude between a first and subsequent peak of a prior or subsequent evoked muscle response caused by stimulating the same motor nerve.

22. The method of claim 21, wherein the prior or subsequent evoked muscle response is produced during a repeated train of stimuli to the motor nerve.

23. The method of claim 22, wherein the repeated train of stimuli includes four stimuli, each stimulus capable of causing an evoked muscle response.

24. The method of claim 23, wherein one or more of the four stimuli do not produce a corresponding evoked muscle response.

25. The method of claims 23, wherein the train of stimuli produces a plurality of evoked muscle responses.

26. The method of claim 25, further comprising identifying a peak of at least one of the plurality of evoked muscle responses.

27. The method of claim 23, wherein the determined amplitude is the peak-to-peak amplitude of the evoked muscle response caused by the fourth stimulation.

28. The method of claim 27, wherein the determined amplitude has a value of zero.

29. The method of claim 27, wherein the comparison of the determined amplitude corresponding to the first stimulus and the amplitude corresponding to the fourth stimulus includes determining a ratio of the amplitude corresponding to the fourth stimulus and the amplitude corresponding to the first stimulus.

30. The method of claim 29, wherein the determined amplitude of the fourth stimulus has a value of zero and the ratio is zero.

31. The method of claim 30, further comprising determining if there was an evoked muscle response corresponding to the first, second and third stimuli.

32. The method of claim 31, further comprising stimulating the motor nerve in a tetanic protocol if there is no evoked muscle response corresponding to the first, second and third stimuli.

33. The method of claim 32, further comprising:
a. stimulating the motor nerve with a plurality of temporally spaced stimuli subsequent to stimulating the motor nerve with the tetanic protocol;
b. recording for an evoked muscle response related to each temporally spaced subsequent stimulation; and
c. identifying the number of evoked muscle responses produced by temporally spaced subsequent stimuli.

34. The method of claim 22, wherein the repeated train of stimuli is produced during a tetanic stimulation protocol of the motor nerve.

35. The method of claim 20, wherein each first peak identified is the peak having the largest value in its corresponding evoked muscle response.

36. The method of claim 35, wherein each subsequent peak identified is the peak subsequent to the first peak and having the largest value of the opposite polarity in its corresponding evoked muscle response.

37-41. (canceled)

42. A method for assessing neuromuscular blockade in a subject having been administered a muscle relaxant agent, comprising:
a. stimulating a motor nerve a first, second, third and fourth time;
b. recording for muscle electrical activity in a muscle innervated by the stimulated motor nerve subsequent to each stimulation of the motor nerve;
c. quantifying the muscle electrical response caused by the first and fourth stimuli; and
d. determining a ratio of the quantified muscle electrical responses caused by the fourth and first stimuli, wherein the ratio indicates a level of neuromuscular blockade in the subject.

43. The method of claim 42, wherein each stimulus is sufficient to cause an evoked muscle response.

44. The method of claim 43, wherein each stimulus is temporally distinct.

45. The method of claim 42, wherein each of the first, second, third and fourth stimulus causes an evoked muscle response.

46-48. (canceled)

49. The method of claim 42, wherein each of the first, second, third and fourth stimulus does not cause an evoked muscle response.

50-52. (canceled)

53. The method of claim 49, wherein the determined ratio is zero.

54. The method of claim 42, wherein the fourth stimulus does not cause an evoked muscle response and the determined ratio is zero.

55. The method of claim 53, further comprising determining if an evoked muscle response was caused by one or more of the third, second or first stimuli.

56. (canceled)

57. (canceled)

58. The method of claim 55, further comprising applying a series of stimuli to the motor nerve using a tetanic stimulation protocol.

59. The method of claim 58, wherein the tetanic stimulation protocol does not cause an evoked muscle response and a ratio for the tetanic stimulation protocol is zero.

60-66. (canceled)

67. A system for assessing neuromuscular blockade in a patient having been administered a muscle relaxant agent, comprising:
a. a stimulator configured to generate a stimulus to a motor nerve at least a first, second, third and fourth time;
b. a patient-stimulus interface configured to supply each stimulus generated by the stimulator to the patient;
c. a patient-recording interface configured to record muscle electrical activity in a muscle innervated by the stimulated motor nerve subsequent to each stimulation of the motor nerve; and
d. at least one processing device configured to quantify the muscle electrical response caused by the first and fourth stimuli and to determine a ratio of the quantified muscle electrical responses caused by the fourth and first stimuli, wherein the ratio indicates a level of neuromuscular blockade in the subject.

68. The system of claim 67, wherein the processing device is further configured to determine if the first, second or third stimuli caused evoked muscle responses.

69. The system of claim 68, wherein the processing device is further configured to cause the stimulator to generate a plurality of stimuli for tetanic stimulation if the first, second, third and fourth stimuli do not cause evoked muscle responses.

70-72. (canceled)

73. The system of claim 67, wherein the at least one processing device is further configured to quantify the muscle electrical response caused by the first and fourth stimuli by identifying a peak of a recorded evoked muscle response and determining the amplitude of the peak from baseline.

74. The system of claim 67, wherein the at least one processing device is further configured to quantify the muscle electrical response caused by the first and fourth stimuli by identifying a peak of the recorded evoked muscle response, identifying a subsequent peak of opposite polarity of the recorded evoked muscle response and determining the peak-to-peak amplitude by determining the difference between the first and subsequent peak.

75-90. (canceled)

91. A system for assessing neuromuscular blockade in a subject having been administered a muscle relaxant agent, comprising:
a. a stimulator configured for stimulating a motor nerve with a plurality temporally distinct stimuli;
b. a recorder for recording for a muscle response in the muscle innervated by the stimulated motor nerve; and
c. at least one processor for evaluating the muscle response following the application of a plurality of the stimuli to provide an assessment of neuromuscular blockade in the subject.

92. (canceled)

93. The system of claim 91, wherein the at least one processor is configured to evaluate the muscle response by determining a ratio of the muscle response resulting from a subsequent and first stimulus.

94-96. (canceled)

97. The system of claim 93, wherein the at least one processor is configured to determine the ratio from the muscle response related to the fourth stimulus and the muscle response related to the first stimulus.

98. The system of claim 93, wherein the at least one processor is configured to determine the ratio from the muscle response related to a fifth or greater stimulus and the muscle response related to the first stimulus.

99-110. (canceled)
